(19) United States
(12) Patent Application Publication
(10) Pub. No.: US 2022/0112172 A1
(43) Pub. Date: Apr. 14, 2022

(54) PROCESS FOR PRODUCING DIOL

(71) Applicant: Covestro Intellectual Property GmbH & Co. KG, Leverkusen (DE)

(72) Inventors: Hartmut Nefzger, Pulheim (DE); Jakob Marbach, Köln (DE); Harald Knaup, Neuss (DE)

(21) Appl. No.: 17/417,604

(22) PCT Filed: Jan. 9, 2020
(86) PCT No.: PCT/EP2020/050466

§ 371 Date: Jun. 23, 2021

(30) Foreign Application Priority Data
Jan. 15, 2019 (EP) 19151747.3

(51) Int. Cl. C07D 317/36; C08G 65/26; C08G 64/40; C08G 64/34

(52) U.S. Cl. CPC C07D 317/36; C08G 65/2663; C08G 64/34; C08G 64/406; C08G 65/2609

(57) ABSTRACT

The invention provides a process for producing diol, characterized in that the process comprises the steps of (1-i) addition of alkylene oxide and carbon dioxide to an H-functional starter substance in the presence of a catalyst to obtain polyether carbonate polyol and cyclic carbonate, (1-ii) separation of the cyclic carbonate from the resulting reaction mixture from step (1-i), (1-iii) hydrolytic cleavage of the cyclic carbonate separated from step (1-ii) into carbon dioxide and diol, (1-iv) optionally distillative purification of the diol from step (1-iii), wherein (η) to the cyclic carbonate from step (1-ii) and/or to the diol a Lewis or Brønsted acid, excluding carboxylic acids having a pKa of >3.0, and optionally water are added and the reaction mixture obtained is optionally neutralized.

### CROSS REFERENCE TO RELATED APPLICATIONS

This application is a U.S. national stage application, filed under 35 U.S.C. § 371, of International Application No. PCT/EP2020/050466, which was filed on Jan. 9, 2020, and which claims priority to European Patent Application No. 19151747.3, which was filed on Jan. 15, 2019. The contents of each are hereby incorporated by reference into this specification.

### FIELD

The present invention relates to a process for preparing diol from cyclic carbonate which is obtained from the preparation of polyethercarbonate polyols.

### BACKGROUND

The preparation of polyethercarbonate polyols by catalytic reaction of alkylene oxides (epoxides) and carbon dioxide in the presence of H-functional starter substances (“starters”) has been the subject of intensive study for more than 40 years (e.g. Inoue et al., Copolymerization of Carbon Dioxide and Epoxide with Organometallic Compounds; Die Makromolekulare Chemie [Macromolecular Chemistry] 130, 210-220, 1969). This reaction is shown in schematic form in scheme (I), where R is an organic radical such as alkyl, alkylaryl or aryl, each of which may also contain heteroatoms, for example O, S, Si, etc., and where e, f and g are each integers, and where the product shown here in scheme (I) for the polyethercarbonate polyol should merely be understood in such a way that blocks having the structure shown may in principle be present in the polyethercarbonate polyol obtained, but the sequence, number and length of the blocks and the OH functionality of the starter may vary and is not restricted to the polyethercarbonate polyol shown in scheme (I). This reaction (see scheme (I)) is highly advantageous from an environmental standpoint since this reaction is the conversion of a greenhouse gas such as CO₂ to a polymer and hence replaces, i.e. saves, a corresponding amount of alkylene oxide. The by-product formed is the cyclic carbonate shown in scheme (I) (by way of example propylene carbonate for R=CH₃, also referred to as cPC hereafter, or ethylene carbonate for R=H, also referred to as cEC hereafter).

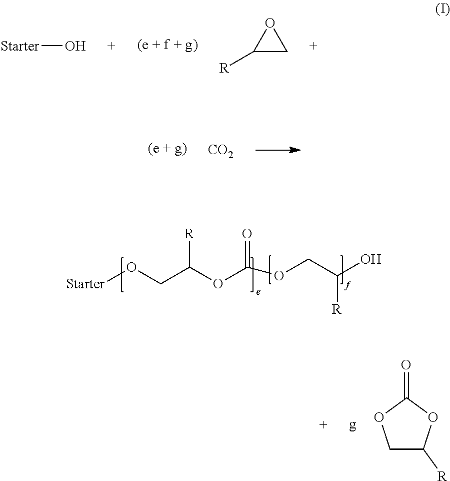

For the utilization of the cyclic carbonates obtained as by-products, for example, the reaction of the cyclic carbonates with amines is disclosed. The resultant urethanediols (WO 2015/075057 A1 and WO 2015/091246 A1) or diurethanediols (WO 2016/188838 A1 and WO 2016/188992 A1) are used as H-functional starter substance for preparation of polyols, for example polyether polyols or polycarbonate polyols.

The preparation of diols from cyclic carbonates is known, for example, from WO 2009/071651 A1 and U.S. Pat. No. 6,080,897. The processes disclose that it is possible by addition of CO₂ onto alkylene oxide to prepare cyclic carbonate and to obtain a diol by subsequent hydrolysis in the presence of water and a catalyst. The preparation of the cyclic carbonate is performed directly in the presence of water. Disadvantages of these processes include the energy expenditure and technical complexity for preparation of the cyclic carbonate and the use of fresh alkylene oxide.

The breakdown of spiro compounds, for example spiro-orthocarbonate, by acidic hydrolysis in THF is described in H. Tagoshi, T. Endo: Bull. Chem. Soc. Jpn., 62, 945-947, 1989.

It was therefore an object of the present invention to maximize the yield based on the alkylene oxide used in the preparation of polyethercarbonate polyols, i.e. to send the compounds obtained as by-products to a physical utilization in a polyol application.

### SUMMARY

This object was achieved according to the invention by a process for preparing diol, characterized in that the process comprises the steps of
(1-i) adding alkylene oxide and carbon dioxide onto an H-functional starter substance in the presence of a catalyst to obtain polyethercarbonate polyol and cyclic carbonate,
(1-ii) separating the cyclic carbonate from the resulting reaction mixture from step (1-i),
(1-iii) hydrolyzing the cyclic carbonate separated from step (1-ii) to carbon dioxide and diol,
(1-iv) optionally purifying the diol from step (1-iii) by distillation,
wherein
(η) a Lewis or Brønsted acid, excluding carboxylic acids having a pKa of >3.0, and optionally water are added to the cyclic carbonate from step (1-ii) and/or the diol, and the reaction mixture obtained is optionally neutralized.

Preferably, in a second process stage
(2-i) polyol is obtained by
   a) adding alkylene oxide and optionally carbon dioxide, cyclic carboxylic anhydride and/or cyclic esters onto the diol that results from the first process stage and optionally further H-functional starter substance
   or
   b) reacting carboxylic acid, cyclic carboxylic anhydride, acyclic ester and/or cyclic ester with the diol that results from the first process stage and optionally further alcohols.

It has been found that, surprisingly, the spiro-orthocarbonate present in the cyclic carbonate was convertible without using a solvent. In a preferred embodiment of the process of the invention, it was simultaneously possible to keep the proportion of the DMD (dimethyldioxane) by-product at a low level. This process leads not only to complete utilization of the alkylene oxide used for the preparation of polyethercarbonate polyols, but also leads to a less costly and more sustainable process through the avoidance of further workup steps for removing by-products formed.

### DETAILED DESCRIPTION

Step (1-i):

In step (1-i), the addition of alkylene oxide and carbon dioxide onto an H-functional starter substance in the presence of a catalyst affords polyethercarbonate polyol and cyclic carbonate, preferably by a process characterized in that
(α) a reactor is charged with a portion of H-functional starter substance and/or a suspension medium having no H-functional groups, optionally together with catalyst,
(β) a DMC catalyst is activated if appropriate by adding a portion (based on the total amount of alkylene oxide used in the activation and copolymerization) of alkylene oxide to the mixture from step (α), where this addition of a portion of alkylene oxide can optionally be effected in the presence of CO₂, in which case the temperature spike (“hotspot”) that occurs owing to the exothermic chemical reaction that follows and/or a pressure drop in the reactor is awaited in each case, and where step (β) for activation may also be effected repeatedly,
(γ) an H-functional starter substance, alkylene oxide and optionally a suspension medium having no H-functional groups and/or carbon dioxide are metered into the reactor during the reaction,
(δ) the reaction mixture removed continuously in step (γ) is optionally transferred into a postreactor in which, by way of a postreaction, the content of free alkylene oxide in the reaction mixture is reduced.

The catalyst used in step (1-i) is at least one compound that catalyzes the addition of alkylene oxide and carbon dioxide, preferably at least one of double metal cyanide catalyst or at least one metal complex catalyst based on the metals zinc and/or cobalt, more preferably a double metal cyanide catalyst.

(α):

The portion of the H-functional starter substance used in (α) may contain component K, preferably in an amount of at least 100 ppm, more preferably of 100 to 10 000 ppm.

In the process, the reactor can first be charged with a portion of H-functional starter substance and/or a suspension medium having no H-functional groups. Subsequently, any amount of catalyst required for the polyaddition is added to the reactor. The sequence of addition is not critical. It is also possible first to charge the reactor with the catalyst and then with a portion of H-functional starter substance. It is alternatively also possible first to suspend the catalyst in a portion of H-functional starter substance and then to charge the reactor with the suspension.

In a preferred embodiment of the invention, in step (α), the reactor is charged with an H-functional starter substance, optionally together with catalyst, without including any suspension medium not containing H-functional groups in the reactor charge.

The catalyst is preferably used in an amount such that the content of catalyst in the resulting reaction product is 10 to 10 000 ppm, more preferably 20 to 5000 ppm, and most preferably 50 to 500 ppm.

In a preferred embodiment, inert gas (for example argon or nitrogen), an inert gas/carbon dioxide mixture or carbon dioxide is introduced into the resulting mixture of (a) a portion of H-functional starter substance and (b) catalyst at a temperature of 90° C. to 150° C., more preferably of 100° C. to 140° C., and at the same time a reduced pressure (absolute) of 10 mbar to 800 mbar, more preferably of 50 mbar to 200 mbar, is applied.

In an alternative preferred embodiment, the resulting mixture of (a) a portion of H-functional starter substance and (b) catalyst is contacted at least once, preferably three times, at a temperature of 90° C. to 150° C., more preferably of 100° C. to 140° C., with 1.5 bar to 10 bar (absolute), more preferably 3 bar to 6 bar (absolute), of an inert gas (for example argon or nitrogen), an inert gas/carbon dioxide mixture or carbon dioxide and then the gauge pressure is reduced in each case to about 1 bar (absolute).

The catalyst can be added in solid form or as a suspension in suspension medium which comprises no H-functional groups in H-functional starter substance or in a mixture thereof.

In a further preferred embodiment, in step (α),
(α-I) a portion of H-functional starter substance is initially charged and
(α-II) the temperature of the portion of H-functional starter substance is brought to 50 to 200° C., preferably 80 to 160° C., more preferably 100 to 140° C., and/or the pressure in the reactor is lowered to less than 500 mbar, preferably 5 mbar to 100 mbar, in the course of which an inert gas stream (for example of argon or nitrogen), an inert gas/carbon dioxide stream or a carbon dioxide stream is optionally passed through the reactor,
   wherein the catalyst is added to the portion of H-functional starter substance in step (α-I) or immediately thereafter in step (α-II).

Step (β):

Step (β) serves to activate the DMC catalyst. This step may optionally be performed under an inert gas atmosphere, under an atmosphere composed of inert gas/carbon dioxide mixture or under a carbon dioxide atmosphere. Activation in the context of this invention refers to a step in which a portion of alkylene oxide is added to the DMC catalyst suspension at temperatures of 90° C. to 150° C. and then the addition of the alkylene oxide is stopped, with observation of evolution of heat caused by a subsequent exothermic chemical reaction, which can lead to a temperature peak (“hotspot”), and of a pressure drop in the reactor caused by the conversion of alkylene oxide and possibly CO₂. The process step of activation is the period from addition of the portion of alkylene oxide, optionally in the presence of CO₂, to the DMC catalyst until evolution of heat occurs. Optionally, the portion of the alkylene oxide can be added to the DMC catalyst in a plurality of individual steps, optionally in the presence of CO₂, and then the addition of the alkylene oxide can be stopped in each case. In this case, the process step of activation comprises the period from the addition of the first portion of alkylene oxide, optionally in the presence of CO₂, to the DMC catalyst until the occurrence of the evolution of heat after addition of the last portion of alkylene oxide. In general, the activation step may be preceded by a step for drying the DMC catalyst and optionally the H-functional starter substance at elevated temperature and/or reduced pressure, optionally with passage of an inert gas through the reaction mixture.

The alkylene oxide (and optionally the carbon dioxide) can in principle be metered in in different ways. The metered addition can be commenced from the vacuum or at a previously chosen supply pressure. The supply pressure is preferably established by introduction of an inert gas (for example nitrogen or argon) or of carbon dioxide, where the pressure (in absolute terms) is 5 mbar to 100 bar, preferably 10 mbar to 50 bar and more preferably 20 mbar to 50 bar.

In one preferred embodiment, the amount of the alkylene oxide used in the activation in step (β) is 0.1% to 25.0% by weight, preferably 1.0% to 20.0% by weight, particularly preferably 2.0% to 16.0% by weight (based on the amount of H-functional starter substance used in step (α)). The alkylene oxide can be added in one step or in two or more portions. Preferably, addition of a portion of the alkylene oxide is followed by interruption of the addition of the alkylene oxide until the occurrence of evolution of heat, and only then is the next portion of alkylene oxide added. A two-stage activation is also preferred (step β), wherein
(β1) in a first activation stage a first portion of alkylene oxide is added under an inert gas atmosphere and
(β2) in a second activation stage a second portion of alkylene oxide is added under a carbon dioxide atmosphere.

Step (γ):

The metered addition of H-functional starter substance, alkylene oxide and optionally a suspension medium having no H-functional groups, and/or of the carbon dioxide can be effected simultaneously or sequentially (in portions); for example, it is possible to add the total amount of carbon dioxide, the amount of H-functional starter substance, of the suspension medium having no H-functional groups and/or the amount of alkylene oxide metered in in step (γ) all at once or continuously. The term “continuously” as used here can be defined as a mode of addition of a reactant such that a concentration of the reactant effective for the copolymerization is maintained, meaning that, for example, the metered addition can be effected with a constant metering rate, with a varying metering rate or in portions.

It is possible, during the addition of the alkylene oxide, the suspension medium having no H-functional groups and/or H-functional starter substance, to increase or lower the CO₂ pressure gradually or stepwise or to leave it constant. The total pressure is preferably kept constant during the reaction by metered addition of further carbon dioxide. The metered addition of the alkylene oxide, the suspension medium having no H-functional groups and/or H-functional starter substance is effected simultaneously or sequentially with respect to the metered addition of carbon dioxide. It is possible to meter in the alkylene oxide at a constant metering rate or to increase or lower the metering rate gradually or in steps or to add the alkylene oxide in portions. The alkylene oxide is preferably added to the reaction mixture at a constant metering rate. If two or more alkylene oxides are used for synthesis of the polyethercarbonate polyols, the alkylene oxides can be metered in individually or as a mixture. The metered addition of the alkylene oxide, the suspension medium having no H-functional groups and the H-functional starter substance can be effected simultaneously or sequentially, each via separate feeds (additions) or via one or more feeds, in which case the alkylene oxide, the suspension medium having no H-functional groups and the H-functional starter substance can be metered in individually or as a mixture. It is possible via the manner and/or sequence of metered addition of the H-functional starter substance, the alkylene oxide, the suspension medium having no H-functional groups and/or the carbon dioxide to synthesize random, alternating, block or gradient polyethercarbonate polyols.

It is preferable to use an excess of carbon dioxide based on the calculated amount of carbon dioxide to be incorporated in the polyethercarbonate polyol, since an excess of carbon dioxide is advantageous because of the inertness of carbon dioxide. The amount of carbon dioxide may be determined via the total pressure under the particular reaction conditions. An advantageous total pressure (in absolute terms) for the copolymerization for preparation of the polyethercarbonate polyols has been found to be in the range from 0.01 to 120 bar, preferably 0.1 to 110 bar, more preferably from 1 to 100 bar. It is possible to feed in the carbon dioxide continuously or discontinuously. This depends on how quickly the alkylene oxide is consumed and whether the product is supposed to contain any CO₂-free polyether blocks. The amount of the carbon dioxide (reported as pressure) can likewise vary in the course of addition of the alkylene oxide. CO₂ can also be added to the reactor in solid form and then be converted to the gaseous, dissolved, liquid and/or supercritical state under the chosen reaction conditions.

For the process according to the invention, it has additionally been found that the copolymerization (step (γ)) for preparation of the polyethercarbonate polyols is conducted advantageously at 50° C. to 150° C., preferably at 60° C. to 145° C., more preferably at 70° C. to 140° C. and most preferably at 90° C. to 130° C. If temperatures are set below 50° C., the reaction generally becomes very slow. At temperatures above 150° C., the amount of unwanted by-products rises significantly.

Catalyst may optionally likewise be metered in in step (γ). The metered addition of the alkylene oxide, H-functional starter substance, the suspension medium having no H-functional groups and the catalyst can be effected via separate or combined metering points. In a preferred embodiment, alkylene oxide, H-functional starter substance and any suspension medium having no H-functional groups are metered into the reaction mixture continuously via separate metering points. This addition of H-functional starter substance and the suspension medium having no H-functional groups can be effected as a continuous metered addition into the reactor or in portions.

Steps (α), (β) and (γ) may be performed in the same reactor or may each be performed separately in different reactors. Particularly preferred reactor types are: tubular reactors, stirred tanks, loop reactors.

Polyethercarbonate polyols can be prepared in a stirred tank, in which case the stirred tank, according to the embodiment and mode of operation, is cooled via the reactor jacket, internal cooling surfaces and/or cooling surfaces within a pumped circulation system. Both in semi-batchwise application, in which the product is not removed until after the end of the reaction, and in continuous application, in which the product is removed continuously, particular attention should be paid to the metering rate of the alkylene oxide. This should be set such that, in spite of the inhibiting action of the carbon dioxide, the alkylene oxide reacts sufficiently quickly. The concentration of free alkylene oxide in the reaction mixture during the activation step (step β) is preferably >0% to 100% by weight, more preferably >0% to 50% by weight, most preferably >0% to 20% by weight (based in each case on the weight of the reaction mixture). The concentration of free alkylene oxide in the reaction mixture during the reaction (step γ) is preferably >0% to 40% by weight, more preferably >0% to 25% by weight, very preferably >0% to 15% by weight (based in each case on the weight of the reaction mixture).

In a preferred embodiment, the mixture containing activated DMC catalyst that results from steps (α) and (β) is reacted further in the same reactor with alkylene oxide, H-functional starter substance, any suspension medium having no H-functional groups, and carbon dioxide. In a further preferred embodiment, the mixture containing activated DMC catalyst that results from steps (α) and (β) is reacted further with alkylene oxide, H-functional starter substance, any suspension medium having no H-functional groups, and carbon dioxide in another reaction vessel (for example a stirred tank, tubular reactor or loop reactor).

When conducting the reaction in a tubular reactor, the mixture containing activated DMC catalyst that results from the steps (α) and (β), H-functional starter substance, alkylene oxide, any suspension medium having no H-functional groups, and carbon dioxide are pumped continuously through a tube. The molar ratios of the co-reactants vary according to the desired polymer. In a preferred embodiment, carbon dioxide is metered in here in its liquid or supercritical form, in order to enable optimal miscibility of the components. Advantageously, mixing elements for better mixing of the coreactants are installed, as sold, for example, by Ehrfeld Mikrotechnik BTS GmbH, or mixer-heat exchanger elements which simultaneously improve the mixing and heat removal.

Loop reactors can likewise be used for preparation of polyethercarbonate polyols. These generally include reactors with recycling of matter, for example a jet loop reactor, which can also be operated continuously, or a tubular reactor designed in the form of a loop with suitable apparatuses for circulation of the reaction mixture, or a loop of a plurality of series-connected tubular reactors. The use of a loop reactor is therefore advantageous especially because backmixing can be achieved here, such that it is possible to keep the concentration of free alkylene oxide in the reaction mixture within the optimal range, preferably in the range from >0% to 40% by weight, more preferably >0% to 25% by weight, most preferably >0% to 15% by weight (based in each case on the weight of the reaction mixture).

Preferably, the polyethercarbonate polyols are prepared in a continuous process which comprises both a continuous copolymerization and a continuous addition of H-functional starter substance and any suspension medium having no H-functional groups.

The invention therefore also provides a process wherein, in step (γ), H-functional starter substance, alkylene oxide, any suspension medium having no H-functional groups and DMC catalyst are metered continuously into the reactor in the presence of carbon dioxide (“copolymerization”) and wherein the resulting reaction mixture (comprising polyethercarbonate polyol and cyclic carbonate) is removed continuously from the reactor. It is preferable when in step (γ), the DMC catalyst is continuously added in the form of a suspension in H-functional starter substance.

For example, for the continuous process for preparing the polyethercarbonate polyols, a mixture containing DMC catalyst is prepared, then, in step (γ),
(γ1) a portion each of H-functional starter substance, alkylene oxide and carbon dioxide are metered in to initiate the copolymerization, and
(γ2) during the progress of the copolymerization, the remaining amount of each of DMC catalyst, H-functional starter substance, any suspension medium having no H-functional groups, and alkylene oxide is metered in continuously in the presence of carbon dioxide, with simultaneous continuous removal of resulting reaction mixture from the reactor.

In step (γ), the catalyst is preferably added in the form of a suspension in H-functional starter substance, the amount preferably being chosen such that the content of catalyst in the resulting reaction product is 10 to 10 000 ppm, more preferably 20 to 5000 ppm, and most preferably 50 to 500 ppm.

Preferably, steps (α) and (β) are performed in a first reactor, and the resulting reaction mixture is then transferred into a second reactor for the copolymerization of step (γ). However, it is also possible to perform steps (α), (β) and (γ) in one reactor.

The term “continuously” used here can be defined as the mode of addition of a relevant catalyst or reactant such that an essentially continuously effective concentration of the catalyst or the reactant is maintained. The catalyst can be fed in in a truly continuous manner or in relatively closely spaced increments. Continuous addition of H-functional starter substance and continuous addition of the suspension medium having no H-functional groups can likewise be truly continuous or in increments. There would be no departure from the present process in adding a catalyst or reactants incrementally such that the concentration of the materials added drops essentially to zero for a period of time before the next incremental addition. However, it is preferable that the catalyst concentration is kept essentially at the same concentration during the main portion of the progression of the continuous reaction, and that H-functional starter substance is present during the main portion of the copolymerization process. An incremental addition of catalyst and/or reactant which does not substantially influence the nature of the product is nevertheless “continuous” in that sense in which the term is being used here. It is possible, for example, to provide a recycling loop in which a portion of the reacting mixture is recycled to a prior point in the process, thus smoothing out discontinuities caused by incremental additions.

Step (δ)

Optionally, in a step (δ), the reaction mixture in step (γ) can be transferred into a postreactor in which, by way of a postreaction, the content of free alkylene oxide in the reaction mixture is reduced. The postreactor used may, for example, be a tubular reactor, a loop reactor or a stirred tank.

The pressure in this postreactor is preferably at the same pressure as in the reaction apparatus in which reaction step (γ) is performed. The pressure in the downstream reactor can, however, also be selected at a higher or lower level. In a further preferred embodiment, the carbon dioxide, after reaction step (γ), is fully or partly released and the downstream reactor is operated at standard pressure or a slightly elevated pressure. The temperature in the downstream reactor is preferably 50 to 150° C. and more preferably 80 to 140° C.

The polyethercarbonate polyols obtained have a functionality, for example, of at least 1, preferably of 1 to 8, more preferably of 1 to 6 and most preferably of 2 to 4. The molecular weight is preferably 400 to 10 000 g/mol and more preferably 500 to 6000 g/mol.

Alkylene Oxide

In general, it is possible to use alkylene oxides (epoxides) having 2-24 carbon atoms for the process. The alkylene oxides having 2-24 carbon atoms are, for example, one or more compounds selected from the group consisting of ethylene oxide, propylene oxide, 1-butene oxide, 2,3-butene oxide, 2-methyl-1,2-propene oxide (isobutene oxide), 1-pentene oxide, 2,3-pentene oxide, 2-methyl-1,2-butene oxide, 3-methyl-1,2-butene oxide, 1-hexene oxide, 2,3-hexene oxide, 3,4-hexene oxide, 2-methyl-1,2-pentene oxide, 4-methyl-1,2-pentene oxide, 2-ethyl-1,2-butene oxide, 1-heptene oxide, 1-octene oxide, 1-nonene oxide, 1-decene oxide, 1-undecene oxide, 1-dodecene oxide, 4-methyl-1,2-pentene oxide, butadiene monoxide, isoprene monoxide, cyclopentene oxide, cyclohexene oxide, cycloheptene oxide, cyclooctene oxide, styrene oxide, methylstyrene oxide, pinene oxide, mono- or polyepoxidized fats as mono-, di- and triglycerides, epoxidized fatty acids, C₁-C₂₄ esters of epoxidized fatty acids, epichlorohydrin, glycidol, and derivatives of glycidol, for example methyl glycidyl ether, ethyl glycidyl ether, 2-ethylhexyl glycidyl ether, allyl glycidyl ether, glycidyl methacrylate and epoxy-functional alkoxysilanes, for example 3-glycidyloxypropyltrimethoxysilane, 3-glycidyloxypropyltriethoxysilane, 3-glycidyloxypropyltripropoxysilane, 3-glycidyloxypropylmethyldimethoxysilane, 3-glycidyloxypropylethyldiethoxysilane, 3-glycidyloxypropyltriisopropoxysilane. The alkylene oxide used is preferably ethylene oxide and/or propylene oxide, especially propylene oxide. In the process according to the invention, the alkylene oxide used may also be a mixture of alkylene oxides.

H-Functional Starter Substance

Suitable H-functional starter substances (“starters”) employed may be compounds having alkoxylation-active hydrogen atoms and having a molar mass of 18 to 4500 g/mol, preferably of 62 to 500 g/mol and more preferably of 62 to 182 g/mol.

Alkoxylation-active groups having active H atoms are, for example, —OH, —NH₂ (primary amines), —NH— (secondary amines), —SH, and —CO₂H, preferably —OH and —NH₂, more preferably —OH. H-functional starter substances used are, for example, one or more compounds selected from the group consisting of mono- or polyhydric alcohols, polyfunctional amines, polyfunctional thiols, amino alcohols, thio alcohols, hydroxy esters, polyether polyols, polyester polyols, polyester ether polyols, polyethercarbonate polyols, polycarbonate polyols, polycarbonates, polyethyleneimines, polyetheramines, polytetrahydrofurans (e.g. PolyTHF® from BASF), polytetrahydrofuran amines, polyether thiols, polyacrylate polyols, castor oil, the mono- or diglyceride of ricinoleic acid, monoglycerides of fatty acids, chemically modified mono-, di- and/or triglycerides of fatty acids, and C₁-C₂₄ alkyl fatty acid esters containing an average of at least 2 OH groups per molecule. By way of example, the C₁-C₂₄-alkyl fatty acid esters containing an average of at least 2 OH groups per molecule are commercial products such as Lupranol Balance® (from BASF AG), Merginol® products (from Hobum Oleochemicals GmbH), Sovermol® products (from Cognis Deutschland GmbH & Co. KG) and Soyol® products (from USSC Co.).

Monofunctional starter substances used may be alcohols, amines, thiols and carboxylic acids. Monofunctional alcohols used may be: methanol, ethanol, 1-propanol, 2-propanol, 1-butanol, 2-butanol, tert-butanol, 3-buten-1-ol, 3-butyn-1-ol, 2-methyl-3-buten-2-ol, 2-methyl-3-butyn-2-ol, propargyl alcohol, 2-methyl-2-propanol, 1-tert-butoxy-2-propanol, 1-pentanol, 2-pentanol, 3-pentanol, 1-hexanol, 2-hexanol, 3-hexanol, 1-heptanol, 2-heptanol, 3-heptanol, 1-octanol, 2-octanol, 3-octanol, 4-octanol, phenol, 2-hydroxybiphenyl, 3-hydroxybiphenyl, 4-hydroxybiphenyl, 2-hydroxypyridine, 3-hydroxypyridine, 4-hydroxypyridine. Useful monofunctional amines include: butylamine, tert-butylamine, pentylamine, hexylamine, aniline, aziridine, pyrrolidine, piperidine, morpholine. Monofunctional thiols used may be: ethanethiol, 1-propanethiol, 2-propanethiol, 1-butanethiol, 3-methyl-1-butanethiol, 2-butene-1-thiol, thiophenol. Monofunctional carboxylic acids include: formic acid, acetic acid, propionic acid, butyric acid, fatty acids such as stearic acid, palmitic acid, oleic acid, linoleic acid, linolenic acid, benzoic acid, acrylic acid.

Polyhydric alcohols suitable as H-functional starter substance are, for example, dihydric alcohols (for example ethylene glycol, diethylene glycol, propylene glycol, dipropylene glycol, propane-1,3-diol, 2-methylpropane-1,3-diol, butane-1,4-diol, butene-1,4-diol, butyne-1,4-diol, neopentyl glycol, pentane-1,5-diol, methylpentanediols (for example 3-methylpentane-1,5-diol), hexane-1,6-diol, octane-1,8-diol, decane-1,10-diol, dodecane-1,12-diol, bis(hydroxymethyl)cyclohexanes (for example 1,4-bis(hydroxymethyl)cyclohexane), triethylene glycol, tetraethylene glycol, polyethylene glycols, dipropylene glycol, tripropylene glycol, polypropylene glycols, dibutylene glycol and polybutylene glycols); trihydric alcohols (for example trimethylolpropane, glycerol, trishydroxyethyl isocyanurate, castor oil); tetrahydric alcohols (for example pentaerythritol); polyalcohols (for example sorbitol, hexitol, sucrose, starch, starch hydrolyzates, cellulose, cellulose hydrolyzates, hydroxy-functionalized fats and oils, in particular castor oil), and all modification products of these aforementioned alcohols with different amounts of ε-caprolactone.

The H-functional starter substance may also be selected from the substance class of the polyether polyols having a molecular weight Mₙ in the range from 18 to 4500 g/mol and a functionality of 2 to 3. Preference is given to polyether polyols formed from repeating ethylene oxide and propylene oxide units, preferably having a proportion of propylene oxide units of 35% to 100%, more preferably having a proportion of propylene oxide units of 50% to 100%. These may be random copolymers, gradient copolymers, alternating copolymers or block copolymers of ethylene oxide and propylene oxide.

The H-functional starter substance may also be selected from the substance class of the polyester polyols. The polyester polyols used are at least difunctional polyesters. Polyester polyols preferably consist of alternating acid and alcohol units. Acid components used are, for example, succinic acid, maleic acid, maleic anhydride, adipic acid, phthalic anhydride, phthalic acid, isophthalic acid, terephthalic acid, tetrahydrophthalic acid, tetrahydrophthalic anhydride, hexahydrophthalic anhydride or mixtures of the acids and/or anhydrides mentioned. Alcohol components used are, for example, ethanediol, propane-1,2-diol, propane-1,3-diol, 2-methylpropane-1,3-diol, butane-1,4-diol, pentane-1,5-diol, neopentyl glycol, hexane-1,6-diol, 1,4-bis(hydroxymethyl)cyclohexane, diethylene glycol, dipropylene glycol, trimethylolpropane, glycerol, pentaerythritol or mixtures of the alcohols mentioned. If the alcohol components used are dihydric or polyhydric polyether polyols, the result is polyester ether polyols which can likewise serve as starter substances for preparation of the polyethercarbonate polyols.

In addition, H-functional starter substance used may be polycarbonatediols which are prepared, for example, by reaction of phosgene, dimethyl carbonate, diethyl carbonate or diphenyl carbonate and difunctional alcohols or polyester polyols or polyether polyols. Examples of polycarbonates may be found, for example, in EP-A 1359177.

In a further embodiment of the invention, polyethercarbonate polyols may be used as H-functional starter substance. More particularly, polyethercarbonate polyols obtainable by the process according to the invention described here are used. For this purpose, these polyethercarbonate polyols used as H-functional starter substance are prepared in a separate reaction step beforehand.

The H-functional starter substance generally has a functionality (i.e. the number of polymerization-active H atoms per molecule) of 1 to 8, preferably of 2 or 3. The H-functional starter substance is used either individually or as a mixture of at least two H-functional starter substances.

More preferably, the H-functional starter substance is at least one of compounds selected from the group consisting of ethylene glycol, propylene glycol, propane-1,3-diol, butane-1,3-diol, butane-1,4-diol, pentane-1,5-diol, 2-methylpropane-1,3-diol, neopentyl glycol, hexane-1,6-diol, octane-1,8-diol, diethylene glycol, dipropylene glycol, glycerol, trimethylolpropane, pentaerythritol, sorbitol, polyethercarbonate polyols having a molecular weight Mₙ in the range from 150 to 8000 g/mol with a functionality of 2 to 3, and polyether polyols having a molecular weight Mₙ in the range from 150 to 8000 g/mol and a functionality of 2 to 3.

In a particularly preferred embodiment, in step (α), the portion of H-functional starter substance is selected from at least one compound of the group consisting of polyethercarbonate polyols having a molecular weight Mₙ in the range from 150 to 8000 g/mol with a functionality of 2 to 3, and polyether polyols having a molecular weight Mₙ in the range from 150 to 8000 g/mol and a functionality of 2 to 3. In a further particularly preferred embodiment, the H-functional starter substance in step (γ) is selected from at least one compound of the group consisting of ethylene glycol, propylene glycol, propane-1,3-diol, butane-1,3-diol, butane-1,4-diol, pentane-1,5-diol, 2-methylpropane-1,3-diol, neopentyl glycol, hexane-1,6-diol, octane-1,8-diol, diethylene glycol, dipropylene glycol, glycerol, trimethylolpropane, pentaerythritol and sorbitol.

The polyethercarbonate polyols are prepared by catalytic addition of carbon dioxide and alkylene oxide onto H-functional starter substance. In the context of the invention, “H-functional” is understood to mean the number of alkoxylation-active hydrogen atoms per molecule of the starter substance.

The H-functional starter substance which is metered continuously into the reactor during the reaction may contain component K.

Suspension Medium

Any suspension medium used contains no H-functional groups. Suitable suspension media having no H-functional groups are all polar aprotic, weakly polar aprotic and nonpolar aprotic solvents, none of which contain any H-functional groups. Suspension media having no H-functional groups that are used may also be a mixture of two or more of these suspension media. The following polar aprotic solvents are mentioned here by way of example: 4-methyl-2-oxo-1,3-dioxolane (also referred to hereinafter as cyclic propylene carbonate or cPC), 1,3-dioxolan-2-one (also referred to hereinafter as cyclic ethylene carbonate or cEC), acetone, methyl ethyl ketone, acetonitrile, nitromethane, dimethyl sulfoxide, sulfolane, dimethylformamide, dimethylacetamide and N-methylpyrrolidone. The group of the nonpolar aprotic and weakly polar aprotic solvents includes, for example, ethers, for example dioxane, diethyl ether, methyl tert-butyl ether and tetrahydrofuran, esters, for example ethyl acetate and butyl acetate, hydrocarbons, for example pentane, n-hexane, benzene and alkylated benzene derivatives (e.g. toluene, xylene, ethylbenzene) and chlorinated hydrocarbons, for example chloroform, chlorobenzene, dichlorobenzene and carbon tetrachloride. Preferred suspension media used having no H-functional groups are 4-methyl-2-oxo-1,3-dioxolane, 1,3-dioxolan-2-one, toluene, xylene, ethylbenzene, chlorobenzene and dichlorobenzene, and mixtures of two or more of these suspension media; particular preference is given to 4-methyl-2-oxo-1,3-dioxolane and 1,3-dioxolan-2-one or a mixture of 4-methyl-2-oxo-1,3-dioxolane and 1,3-dioxolan-2-one.

In step (γ), preferably 2% by weight to 20% by weight, more preferably 5% by weight to 15% by weight and especially preferably 7% by weight to 11% by weight of the suspension medium having no H-functional groups is metered in, based on the sum total of the components metered in in step (γ).

Component K

Compounds suitable as component K are characterized in that they contain at least one phosphorus-oxygen bond. Examples of suitable components K are phosphoric acid and phosphoric salts, phosphoryl halides, phosphoramides, phosphoric esters and salts of the mono- and diesters of phosphoric acid.

In the context of the invention the esters cited as possible components K hereinabove and hereinbelow are to be understood as meaning in each case the alkyl ester, aryl ester and/or alkaryl ester derivatives.

Examples of suitable phosphoric esters include mono-, di- or triesters of phosphoric acid, mono-, di-, tri- or tetraesters of pyrophosphoric acid and mono-, di-, tri-, tetra- or polyesters of polyphosphoric acid with alcohols having 1 to 30 carbon atoms. Examples of compounds suitable as component K include: triethyl phosphate, diethyl phosphate, monoethyl phosphate, tripropyl phosphate, dipropyl phosphate, monopropyl phosphate, tributyl phosphate, dibutyl phosphate, monobutyl phosphate, trioctyl phosphate, tris(2-ethylhexyl) phosphate, tris(2-butoxyethyl) phosphate, diphenyl phosphate, dicresyl phosphate, fructose 1,6-biphosphate, glucose 1-phosphate, bis(dimethylamido)phosphoric chloride, bis(4-nitrophenyl) phosphate, cyclopropylmethyl diethyl phosphate, dibenzyl phosphate, diethyl 3-butenyl phosphate, dihexadecyl phosphate, diisopropyl chlorophosphate, diphenyl phosphate, diphenyl chlorophosphate, 2-hydroxyethyl methacrylate phosphate, mono(4-chlorophenyl) dichlorophosphate, mono(4-nitrophenyl) dichlorophosphate, monophenyl dichlorophosphate, tridecyl phosphate, tricresyl phosphate, trimethyl phosphate, triphenyl phosphate, phosphoric acid tripyrolidide, phosphorus sulfochloride, dimethylamidophosphoric dichloride, methyl dichlorophosphate, phosphoryl bromide, phosphoryl chloride, phosphoryl quinoline chloride calcium salt and O-phosphorylethanolamine, alkali metal and ammonium dihydrogenphosphates, alkali metal, alkaline earth metal and ammonium hydrogenphosphates, alkali metal, alkaline earth metal and ammonium phosphates.

The term “esters of phosphoric acid” (phosphoric esters) is understood also to include the products obtainable by propoxylation of phosphoric acid (available as Exolit® OP 560 for example).

Other suitable components K are phosphonic acid and phosphorous acid and also mono- and diesters of phosphonic acid and mono-, di- and triesters of phosphorous acid and their respective salts, halides and amides.

Examples of suitable phosphonic esters include mono- or diesters of phosphonic acid, alkylphosphonic acids, arylphosphonic acids, alkoxycarbonylalkylphosphonic acids, alkoxycarbonylphosphonic acids, cyanoalkylphosphonic acids and cyanophosphonic acids or mono-, di-, tri- or tetraesters of alkyldiphosphonic acids with alcohols having 1 to 30 carbon atoms. Examples of suitable phosphorous esters include mono-, di- or triesters of phosphorous acid with alcohols having 1 to 30 carbon atoms. This includes, for example, phenylphosphonic acid, butylphosphonic acid, dodecylphosphonic acid, ethylhexylphosphonic acid, octylphosphonic acid, ethylphosphonic acid, methylphosphonic acid, octadecylphosphonic acid and their mono- and dimethyl esters, ethyl esters, butyl esters, ethylhexyl esters or phenyl esters, dibutyl butylphosphonate, dioctyl phenylphosphonate, triethyl phosphonoformate, trimethyl phosphonoacetate, triethyl phosphonoacetate, trimethyl 2-phosphonopropionate, triethyl 2-phosphonopropionate, tripropyl 2-phosphonopropionate, tributyl 2-phosphonopropionate, triethyl 3-phosphonopropionate, triethyl 2-phosphonobutyrate, triethyl 4-phosphonocrotonate, (12-phosphonododecyl)phosphonic acid, phosphonoacetic acid, methyl P,P-bis(2,2,2-trifluoroethyl)phosphonoacetate, trimethylsilyl P,P-diethylphosphonoacetate, tert-butyl P,P-dimethylphosphonoacetate, P,P-dimethyl phosphonoacetate potassium salt, P,P-dimethylethyl phosphonoacetate, 16-phosphonohexadecanoic acid, 6-phosphonohexanoic acid, N-(phosphonomethyl)glycine, N-(phosphonomethyl)glycine monoisopropylamine salt, N-(phosphonomethyl)iminodiacetic acid, (8-phosphonooctyl)phosphonic acid, 3-phosphonopropionic acid, 11-phosphonoundecanoic acid, pinacol phosphonate, trilauryl phosphite, tris(3-ethyloxethanyl-3-methyl) phosphite, heptakis(dipropylene glycol) phosphite, 2-cyanoethyl bis(diisopropylamido)phosphite, methyl bis(diisopropylamido)phosphite, dibutyl phosphite, dibenzyl (diethylamido)phosphite, di-tert-butyl (diethylamido)phosphite, diethyl phosphite, diallyl (diisopropylamido)phosphite, dibenzyl (diisopropylamido)phosphite, di-tert-butyl (diisopropylamido)phosphite, dimethyl (diisopropylamido)phosphite, dibenzyl (dimethylamido)phosphite, dimethyl phosphite, trimethylsilyl dimethylphosphite, diphenyl phosphite, methyl dichlorophosphite, mono(2-cyanoethyl) diisopropylamidochlorophosphite, o-phenylene chlorophosphite, tributyl phosphite, triethyl phosphite, triisopropyl phosphite, triphenyl phosphite, tris(tert-butyl-dimethylsilyl) phosphite, tris-1,1,1,3,3,3-hexafluoro-2-propyl phosphite, tris(trimethylsilyl) phosphite, dibenzyl phosphite. The term “esters of phosphorous acid” is also understood to include the products obtainable by propoxylation of phosphorous acid (available as Exolit® OP 550 for example).

Other suitable components K are phosphinic acid, phosphonous acid and phosphinous acid and their respective esters. Examples of suitable phosphinic esters include esters of phosphinic acid, alkylphosphinic acids, dialkylphosphinic acids or arylphosphinic acids with alcohols having 1 to 30 carbon atoms. Examples of suitable phosphonous esters include mono- and diesters of phosphonous acid or arylphosphonous acid with alcohols having 1 to 30 carbon atoms. This includes, for example, diphenylphosphinic acid or 9,10-dihydro-9-oxa-10-phosphaphenanthrene 10-oxide.

The esters of phosphoric acid, phosphonic acid, phosphorous acid, phosphinic acid, phosphonous acid or phosphinous acid suitable as component K are generally obtained by reaction of phosphoric acid, pyrophosphoric acid, polyphosphoric acids, phosphonic acid, alkylphosphonic acids, arylphosphonic acids, alkoxycarbonylalkylphosphonic acids, alkoxycarbonylphosphonic acids, cyanoalkylphosphonic acids, cyanophosphonic acid, alkyldiphosphonic acids, phosphonous acid, phosphorous acids, phosphinic acid, phosphinous acid or the halogen derivatives or phosphorus oxides thereof with hydroxy compounds having 1 to 30 carbon atoms such as methanol, ethanol, propanol, butanol, pentanol, hexanol, heptanol, octanol, nonanol, decanol, dodecanol, tridecanol, tetradecanol, pentadecanol, hexadecanol, heptadecanol, octadecanol, nonadecanol, methoxymethanol, ethoxymethanol, propoxymethanol, butoxymethanol, 2-ethoxyethanol, 2-propoxyethanol, 2-butoxyethanol, phenol, ethyl hydroxy acetate, propyl hydroxy acetate, ethyl hydroxypropionate, propyl hydroxypropionate, ethane-1,2-diol, propane-1,2-diol, 1,2,3-trihydroxypropane, 1,1,1-trimethylolpropane or pentaerythritol.

Phosphine oxides suitable as component K contain one or more alkyl, aryl or aralkyl groups having 1-30 carbon atoms bonded to the phosphorus. Preferred phosphine oxides have the general formula R₃P═O where R is an alkyl, aryl or aralkyl group having 1-20 carbon atoms. Examples of suitable phosphine oxides include trimethylphosphine oxide, tri(n-butyl)phosphine oxide, tri(n-octyl)phosphine oxide, triphenylphosphine oxide, methyldibenzylphosphine oxide and mixtures thereof.

Also suitable as component K are compounds of phosphorus that can form one or more P—O bond(s) by reaction with OH-functional compounds (such as water or alcohols for example). Examples of such compounds of phosphorus that are useful include phosphorus(V) sulfide, phosphorus tribromide, phosphorus trichloride and phosphorus triiodide. It is also possible to employ any desired mixtures of the abovementioned compounds as component K. More preferably, component K is phosphoric acid.

DMC Catalysts

DMC catalysts for use in the homopolymerization of alkylene oxides are known in principle from the prior art (see, for example, U.S. Pat. Nos. 3,404,109, 3,829,505, 3,941,849 and 5,158,922). DMC catalysts, which are described, for example, in U.S. Pat. No. 5,470,813, EP-A 700 949, EP-A 743 093, EP-A 761 708, WO 97/40086, WO 98/16310 and WO 00/47649, have a very high activity and enable the preparation of polyethercarbonate polyols at very low catalyst concentrations, such that there is generally no longer a need to separate the catalyst from the finished product. A typical example is that of the highly active DMC catalysts which are described in EP-A 700 949 and contain not only a double metal cyanide compound (e.g. zinc hexacyanocobaltate(III)) and an organic complex ligand (e.g. tert-butanol) but also a polyether having a number-average molecular weight greater than 500 g/mol.

The DMC catalysts are preferably obtained by
(i) reacting an aqueous solution of a metal salt with the aqueous solution of a metal cyanide salt in the presence of one or more organic complex ligands, e.g. an ether or alcohol, in the first step,
(ii) separating the solid from the suspension obtained from (i) by known techniques (such as centrifugation or filtration) in the second step,
(iii) optionally washing the isolated solid with an aqueous solution of an organic complex ligand (for example by resuspending and subsequent reisolating by filtration or centrifugation) in a third step,
(iv) then drying the solid obtained at temperatures of generally 20-120° C. and at pressures of generally 0.1 mbar to standard pressure (1013 mbar), optionally after pulverizing,
   wherein, in the first step or immediately after the precipitation of the double metal cyanide compound (second step), one or more organic complex ligands, preferably in excess (based on the double metal cyanide compound), and optionally further complex-forming components are added.

The double metal cyanide compounds present in the DMC catalysts are the reaction products of water-soluble metal salts and water-soluble metal cyanide salts.

For example, an aqueous solution of zinc chloride (preferably in excess based on the metal cyanide salt, for example potassium hexacyanocobaltate) and potassium hexacyanocobaltate are mixed and then dimethoxyethane (glyme) or tert-butanol (preferably in excess, based on zinc hexacyanocobaltate) is added to the suspension formed.

Metal salts suitable for preparation of the double metal cyanide compounds preferably have the general formula (II)

M(X)ₙ (II)

where

M is selected from the metal cations Zn²⁺, Fe²⁺, Ni²⁺, Mn²⁺, Co²⁺, Sr²⁺, Sn²⁺, Pb²⁺ and Cu²⁺; M is preferably Zn²⁺, Fe²⁺, Co²⁺ or Ni²⁺,

X are one or more (i.e. different) anions, preferably an anion selected from the group of halides (i.e. fluoride, chloride, bromide, iodide), hydroxide, sulfate, carbonate, cyanate, thiocyanate, isocyanate, isothiocyanate, carboxylate, oxalate and nitrate;

n is 1 when X=sulfate, carbonate or oxalate and

n is 2 when X=halide, hydroxide, carboxylate, cyanate, thiocyanate, isocyanate, isothiocyanate or nitrate,

or suitable metal salts have the general formula (III)

Mᵣ(X)₃ (III)

where

M is selected from the metal cations Fe³⁺, Al³⁺, Co³⁺ and Cr³⁺,

X are one or more (i.e. different) anions, preferably an anion selected from the group of halides (i.e. fluoride, chloride, bromide, iodide), hydroxide, sulfate, carbonate, cyanate, thiocyanate, isocyanate, isothiocyanate, carboxylate, oxalate and nitrate;

r is 2 when X=sulfate, carbonate or oxalate and

r is 1 when X=halide, hydroxide, carboxylate, cyanate, thiocyanate, isocyanate, isothiocyanate or nitrate,

or suitable metal salts have the general formula (IV)

M(X)ₛ (IV)

where

M is selected from the metal cations Mo⁴⁺, V⁴⁺ and W⁴⁺,

X are one or more (i.e. different) anions, preferably an anion selected from the group of halides (i.e. fluoride, chloride, bromide, iodide), hydroxide, sulfate, carbonate, cyanate, thiocyanate, isocyanate, isothiocyanate, carboxylate, oxalate and nitrate;

s is 2 when X=sulfate, carbonate or oxalate and

s is 4 when X=halide, hydroxide, carboxylate, cyanate, thiocyanate, isocyanate, isothiocyanate or nitrate,

or suitable metal salts have the general formula (V)

M(X)ₜ (V)

where

M is selected from the metal cations Mo⁶⁺ and W⁶⁺,

X are one or more (i.e. different) anions, preferably an anion selected from the group of halides (i.e. fluoride, chloride, bromide, iodide), hydroxide, sulfate, carbonate, cyanate, thiocyanate, isocyanate, isothiocyanate, carboxylate, oxalate and nitrate;

t is 3 when X=sulfate, carbonate or oxalate and

t is 6 when X=halide, hydroxide, carboxylate, cyanate, thiocyanate, isocyanate, isothiocyanate or nitrate.

Examples of suitable metal salts are zinc chloride, zinc bromide, zinc iodide, zinc acetate, zinc acetylacetonate, zinc benzoate, zinc nitrate, iron(II) sulfate, iron(II) bromide, iron(II) chloride, iron(III) chloride, cobalt(II) chloride, cobalt(II) thiocyanate, nickel(II) chloride and nickel(II) nitrate. It is also possible to use mixtures of different metal salts.

Metal cyanide salts suitable for preparation of the double metal cyanide compounds preferably have the general formula (VI)

(Y)ₐM′(CN)_{b}(A)_{c} (VI)

where

M′ is selected from one or more metal cations from the group consisting of Fe(II), Fe(III), Co(II), Co(III), Cr(II), Cr(III), Mn(II), Mn(III), Ir(III), Ni(II), Rh(III), Ru(II), V(IV) and V(V); M′ is preferably one or more metal cations from the group consisting of Co(II), Co(III), Fe(II), Fe(III), Cr(III), Ir(III) and Ni(II),

Y is selected from one or more metal cations from the group consisting of alkali metal (i.e. Li⁺, Na⁺, K⁺, Rb⁺) and alkaline earth metal Be²⁺, Mg²⁺, Ca²⁺, Sr²⁺, Ba²⁺),

A is selected from one or more anions from the group consisting of halides (i.e. fluoride, chloride, bromide, iodide), hydroxide, sulfate, carbonate, cyanate, thiocyanate, isocyanate, isothiocyanate, carboxylate, azide, oxalate or nitrate, and

a, b and c are integers, the values for a, b and c being selected such as to ensure the electronic neutrality of the metal cyanide salt; a is preferably 1, 2, 3 or 4; b is preferably 4, 5 or 6; c preferably has the value 0.

Examples of suitable metal cyanide salts are sodium hexacyanocobaltate(III), potassium hexacyanocobaltate(III), potassium hexacyanoferrate(II), potassium hexacyanoferrate(III), calcium hexacyanocobaltate(III) and lithium hexacyanocobaltate(III).

Preferred double metal cyanide compounds present in the DMC catalysts are compounds of the general formula (VII)

Mₓ[M′ₓ,(CN)_{y}]_{z} (VII)

where M is as defined in formula (II) to (V) and

M′ is as defined in formula (VI), and

x, x′, y and z are integers and are chosen so as to ensure the electronic neutrality of the double metal cyanide compound.

Preferably,

x=3, x′=1, y=6 and z=2,

M=Zn(II), Fe(II), Co(II) or Ni(II) and

M′=Co(III), Fe(III), Cr(III) or Ir(III).

Examples of suitable double metal cyanide compounds a) are zinc hexacyanocobaltate(III), zinc hexacyanoiridate(III), zinc hexacyanoferrate(III) and cobalt(II) hexacyanocobaltate(III). Further examples of suitable double metal cyanide compounds can be found, for example, in U.S. Pat. No. 5,158,922 (column 8, lines 29-66). Particular preference is given to using zinc hexacyanocobaltate(III).

The organic complex ligands added in the preparation of the DMC catalysts are disclosed, for example, in U.S. Pat. No. 5,158,922 (see especially column 6, lines 9 to 65), U.S. Pat. Nos. 3,404,109, 3,829,505, 3,941,849, EP-A 700 949, EP-A 761 708, JP H04 145 123, U.S. Pat. No. 5,470,813, EP-A 743 093 and WO-A 97/40086). For example, organic complex ligands used are water-soluble organic compounds having heteroatoms, such as oxygen, nitrogen, phosphorus or sulfur, which can form complexes with the double metal cyanide compound. Preferred organic complex ligands are alcohols, aldehydes, ketones, ethers, esters, amides, ureas, nitriles, sulfides and mixtures thereof. Particularly preferred organic complex ligands are aliphatic ethers (such as dimethoxyethane), water-soluble aliphatic alcohols (such as ethanol, isopropanol, n-butanol, isobutanol, sec-butanol, tert-butanol, 2-methyl-3-buten-2-ol and 2-methyl-3-butyn-2-ol), compounds containing both aliphatic or cycloaliphatic ether groups and aliphatic hydroxyl groups (for example ethylene glycol mono-tert-butyl ether, diethylene glycol mono-tert-butyl ether, tripropylene glycol monomethyl ether and 3-methyl-3-oxetanemethanol). Most preferred organic complex ligands are selected from one or more compounds of the group consisting of dimethoxyethane, tert-butanol, 2-methyl-3-buten-2-ol, 2-methyl-3-butyn-2-ol, ethylene glycol mono-tert-butyl ether and 3-methyl-3-oxetanemethanol.

Optionally used in the preparation of the DMC catalysts are one or more complex-forming component(s) from the compound classes of the polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylic acid-co-maleic acid), polyacrylonitrile, polyalkylacrylates, polyalkylmethacrylates, polyvinyl methyl ether, polyvinyl ethyl ether, polyvinyl acetate, polyvinyl alcohol, poly-N-vinylpyrrolidone, poly(N-vinylpyrrolidone-co-acrylic acid), polyvinyl methyl ketone, poly(4-vinylphenol), poly(acrylic acid-co-styrene), oxazoline polymers, polyalkyleneimines, maleic acid and maleic anhydride copolymers, hydroxyethyl cellulose and polyacetals, or of the glycidyl ethers, glycosides, carboxylic esters of polyhydric alcohols, gallic acids or the salts, esters or amides thereof, cyclodextrins, phosphorus compounds, α,β-unsaturated carboxylic esters or ionic surface- or interface-active compounds.

Preferably, in the preparation of the DMC catalysts, in the first step, the aqueous solutions of the metal salt (e.g. zinc chloride), used in a stoichiometric excess (at least 50 mol %) based on metal cyanide salt (i.e. at least a molar ratio of metal salt to metal cyanide salt of 2.25:1.00), and of the metal cyanide salt (e.g. potassium hexacyanocobaltate) are converted in the presence of the organic complex ligand (e.g. tert-butanol), forming a suspension containing the double metal cyanide compound (e.g. zinc hexacyanocobaltate), water, excess metal salt and the organic complex ligand.

The organic complex ligand may be present in the aqueous solution of the metal salt and/or of the metal cyanide salt or it is added directly to the suspension obtained after precipitation of the double metal cyanide compound. It has been found to be advantageous to mix the metal salt and the metal cyanide salt aqueous solutions and the organic complex ligand by stirring vigorously. Optionally, the suspension formed in the first step is subsequently treated with a further complex-forming component. This complex-forming component is preferably used in a mixture with water and organic complex ligand. A preferred process for performing the first step (i.e. the preparation of the suspension) is effected using a mixing nozzle, more preferably using a jet disperser as described in WO-A 01/39883.

In the second step, the solid (i.e. the precursor of the catalyst) is isolated from the suspension by known techniques, such as centrifugation or filtration.

In a preferred execution variant, the isolated solid is subsequently washed in a third process step with an aqueous solution of the organic complex ligand (for example by resuspension and subsequent reisolation by filtration or centrifugation). In this way, it is possible to remove, for example, water-soluble by-products such as potassium chloride from the catalyst. The amount of the organic complex ligand in the aqueous wash solution is preferably between 40% and 80% by weight, based on the overall solution.

Further complex-forming component is optionally added to the aqueous wash solution in the third step, preferably in the range between 0.5% and 5% by weight, based on the overall solution.

It is also advantageous to wash the isolated solid more than once. Preferably, in a first wash step (iii-1), washing is effected with an aqueous solution of the organic complex ligand (for example by resuspension and subsequently reisolation by filtration or centrifugation), in order in this way to remove, for example, water-soluble by-products, such as potassium chloride, from the catalyst. More preferably, the amount of the organic complex ligand in the aqueous wash solution is between 40% and 80% by weight, based on the overall solution for the first wash step. In the further wash steps (iii-2), either the first wash step is repeated once or more than once, preferably once to three times, or, preferably, a nonaqueous solution, for example a mixture or solution of organic complex ligand and further complex-forming component (preferably in the range between 0.5 and 5% by weight, based on the total amount of the wash solution in step (iii-2)), is used as a wash solution to wash the solid once or more than once, preferably once to three times.

The isolated and optionally washed solid is subsequently dried, optionally after pulverization, at temperatures of generally 20-100° C. and at pressures of generally 0.1 mbar to standard pressure (1013 mbar).

A preferred process for isolation of the DMC catalysts from the suspension by filtration, filtercake washing and drying is described in WO-A 01/80994.

As well as the DMC catalysts based on zinc hexacyanocobaltate (Zn₃[Co(CN)₆]₂) that are used with preference, it is also possible to use other metal complex catalysts based on the metals zinc and/or cobalt that are known to those skilled in the art from the prior art for the copolymerization of epoxides and carbon dioxide for the process according to the invention. This especially includes what are called zinc glutarate catalysts (described, for example, in M. H. Chisholm et al., Macromolecules 2002, 35, 6494), what are called zinc diiminate catalysts (described, for example, in S. D. Allen, J. Am. Chem. Soc. 2002, 124, 14284), what are called cobalt salen catalysts (described, for example, in U.S. Pat. No. 7,304,172 B2, US 2012/0165549 A1), and bimetallic zinc complexes having macrocyclic ligands (described, for example, in M. R. Kember et al., Angew. Chem., Int. Ed., 2009, 48, 931).

Step (1-i) affords, as shown in scheme (I), polyethercarbonate polyol and, according to the alkylene oxide used in step (1-i), cyclic carbonate. The cyclic carbonate is preferably selected from at least one compound from the group consisting of cyclic propylene carbonate and cyclic ethylene carbonate.

Step (1-ii):

Cyclic carbonate is separated in step (1-ii) from the reaction mixture obtained in step (1-i). The cyclic carbonate can be separated off by methods known to the person skilled in the art. Preference is given here to using thermal methods, for example distillation or rectification. Particular preference is given to separating the cyclic carbonate from the reaction mixture from step (1-i) by means of an evaporation device, for example thin-film evaporator or falling film evaporator, a stripping column or a combination thereof. The cyclic carbonate can be separated off here by a single-stage or multistage process, preference being given to a two-stage process. Most preferably, the cyclic carbonate is separated off in a two-stage process by means of a thin-film evaporator or a falling-film evaporator in combination with a stripping column. If an evaporation device is used in step (1-ii), this can be operated at a pressure of 100 mbar or lower, preferably at 1 to 100 mbar, more preferably at 1 to 50 mbar, especially preferably at 2 to 25 mbar, where the temperature of the reaction mixture in step (1-ii) that has been obtained from step (1-i) is preferably 120 to 180° C., more preferably 150 to 170° C. When a stripping column is used, this can be operated under standard pressure or under reduced pressure, for example at a pressure of 1 to 150 mbar, preferably 50 to 120 mbar, while the stripping column can be operated at a temperature of 120 to 180° C., preferably 150 to 170° C.

An apparatus for separation of cyclic carbonate from a mixture comprising polyethercarbonate polyol, is disclosed, for example, in application EP 3 164 443 A1.

The cyclic propylene carbonate thus obtained can be purified by distillation to remove volatile secondary components, for example dimethyldioxanes, prior to step (1-iii).

Step (1-iii):

The cyclic carbonate obtained from step (1-ii) is subsequently used to charge a reactor together with water and a hydrolysis catalyst, heated and hydrolyzed to carbon dioxide and diol. The CO₂ formed is discharged from the reactor and can optionally be reused in step (1-i). The reaction is continued until the evolution of CO₂ ceases. It is likewise possible to transfer further cyclic carbonate from step (1-ii) into the reactor during the reaction.

The molar ratio of cyclic carbonate to water in step (1-iii) is preferably 1:1 to 1:10, more preferably 1:1.2 to 1:6 and especially preferably 1:1.5 to 1:4. The hydrolysis of the cyclic carbonate is preferably conducted at a temperature of at least 40° C., more preferably at at least 80° C.

The hydrolysis catalysts used in step (1-iii) may be the catalytic compounds known for hydrolysis to the person skilled in the art. Examples of such compounds are alkali metal salts, such as alkali metal hydroxides or alkali metal carbonates, alkaline earth metal salts, such as alkaline earth metal hydroxides or alkaline earth metal carbonates, hydrolases or basic ion exchangers. In step (1-iii), the hydrolysis catalyst used is preferably at least one compound selected from the group consisting of alkali metal hydroxides, alkaline earth metal hydroxides and hydrolases. More preferably, the compound is selected from the group of the alkali metal hydroxides, especially preferably from the group consisting of potassium hydroxide and sodium hydroxide. Most preferably, the hydrolysis catalyst used in step (1-iii) is solely potassium hydroxide and/or sodium hydroxide.

Typically 0.05% to 1% by weight, based on the cyclic carbonate used in step (1-iii), of hydrolysis catalyst is used. But it is also possible to use less than 0.05% or more than 1% by weight.

In a particular embodiment, in step (1-iii), the hydrolysis catalyst used is 0.05% to 1% by weight, based on the cyclic carbonate used in step (1-iii), of at least one compound from the group of the alkali metal hydroxides.

In a further particular embodiment, in step (1-iii), the hydrolysis catalyst used is 0.05% to 1% by weight, based on the cyclic carbonate used in step (1-iii), of at least one compound selected from the group consisting of potassium hydroxide and sodium hydroxide.

Step (1-iv):

The diol obtained in step (1-iii) can optionally be purified by distillation. The distillation may be effected under standard pressure or reduced pressure, in which case it is possible to establish a pressure of 150 mbar or lower, preferably 100 mbar or lower, more preferably 50 mbar or lower, especially preferably 20 mbar or lower. Step (1-iv) may be performed in a single-stage or multistage process, with the use of columns, for example randomly packed columns, in the distillation being considered as a multistage process for the purposes of the invention.

The mixture that has been separated from the diol and comprises hydrolysis catalyst may optionally be reused in step (1-iii).

Step (i):

According to the invention, after step (1-ii), (1-iii) or optionally after step (1-iv), step (η) is performed, comprising
(η) the addition of a Lewis or Brønsted acid, excluding carboxylic acids having a pKa of >3.0, and optionally water to the reaction mixture from the preceding step, and optionally subsequent neutralization of the reaction mixture obtained.

This step can be performed at a temperature in the range from 10 to 160° C., preferably from 15 to 140° C., more preferably from 20 to 120° C.; this step can alternatively be performed at higher temperatures.

Lewis or Brønsted acids used may, for example, be HCl, HBr, HI, H₂SO₄, H₂SO₃, H₃PO₄, H₃PO₃, HNO₃, or carboxylic acids having a pKa ≤3.0, such as trifluoroacetic acid, or compounds having sulfonic acid groups, such as toluenesulfonic acid, benzenesulfonic acid, methanesulfonic acid, trifluoromethanesulfonic acid, including ion exchange resins having sulfonic acid groups, metal salts such as SnCl₂, ZnCl₂, AlCl₃, FeCl₃. It is optionally possible to neutralize the reaction mixture obtained, for example with NaHCO₃, NaOH, KOH, Na₂CO₃, K₂CO₃, CaCO₃, Mg(OH)₂, and optionally to perform a filtration after neutralization.

In a further embodiment, water is additionally added to the reaction mixture from the preceding step.

Step (2-i):

In a second process stage, polyol is preferably obtained by
a) adding alkylene oxide and optionally carbon dioxide, cyclic carboxylic anhydride and/or cyclic esters onto the diol that results from the first process stage and optionally further H-functional starter substance
or
b) reacting carboxylic acid, cyclic carboxylic anhydride, acyclic esters and/or cyclic ester with the diol that results from the first process stage and optionally further alcohols.

Polyols obtained are, for example, polyether polyols, polyester polyols, polyethercarbonate polyols or polyetherester polyols. The polyol is preferably at least one compound selected from the group consisting of polyether polyol, polyester polyol, and polyethercarbonate polyol.

Polyethercarbonate polyols are obtained by adding alkylene oxide and carbon dioxide onto the diol obtained from the first process stage and optionally further H-functional starter substance in the presence of a catalyst. Preference is given to obtaining polyethercarbonate polyols by the process described in step (1-i), where the H-functional starter substance used contains the diol obtained from the first process step.

The polyether polyols prepared according to the invention are obtained by adding alkylene oxide onto the diol that results from the first process stage and any further H-functional starter substance by preparation methods known to the person skilled in the art. The compounds used as alkylene oxide and any further H-functional starter substance have already been described in step (1-i). Preferably, as well as the diol obtainable from the first process step, additional H-functional starter substances used are di- or polyhydric alcohols, such as ethanediol, propane-1,2- and -1,3-diol, diethylene glycol, dipropylene glycol, butane-1,4-diol, hexane-1,6-diol, triethanolamine, bisphenol, glycerol, trimethylolpropane, pentaerythritol, sorbitol or sucrose.

Catalysts used for the preparation of polyether polyols in the second process step may, for example, be alkali metal hydroxides, such as sodium hydroxide or potassium hydroxide, alkali metal alkoxides, such as sodium methoxide, sodium ethoxide or potassium ethoxide or potassium isopropoxide, or aminic alkoxylation catalysts, such as dimethylethanolamine (DMEOA), imidazole and/or imidazole derivatives, or DMC catalysts, as described in step (1-i).

The polyester polyols may, for example, be polycondensates of the diol obtained from the first process stage and any further alcohols, having 2 to 12 carbon atoms, preferably having 2 to 6 carbon atoms, and carboxylic acids, cyclic carboxylic hydrides, acyclic esters and/or cyclic esters. Carboxylic acids are, for example, polycarboxylic acids, for example di-, tri- or even tetracarboxylic acids, or hydroxycarboxylic acids; preference is given to using aromatic and/or aliphatic dicarboxylic acids. Instead of the free polycarboxylic acids, it is also possible to use the corresponding polycarboxylic anhydrides or corresponding polycarboxylic esters of lower alcohols for preparing the polyesters. The starting materials used for preparation of polyester polyols may also be of biological origin and/or may be obtained by fermentative methods.

Useful carboxylic acids especially include: succinic acid, glutaric acid, adipic acid, suberic acid, azelaic acid, sebacic acid, decanedicarboxylic acid, tetrahydrophthalic acid, hexahydrophthalic acid, cyclohexanedicarboxylic acid, tetrachlorophthalic acid, itaconic acid, malonic acid, 2-methylsuccinic acid, 3,3-diethylglutaric acid, 2,2-dimethylsuccinic acid, dodecanedioic acid, endomethylenetetrahydrophthalic acid, dimer fatty acid, trimer fatty acid, citric acid, trimellitic acid, benzoic acid, trimellitic acid, maleic acid, fumaric acid, phthalic acid, isophthalic acid and terephthalic acid. It is likewise possible to use derivatives of these carboxylic acids, for example dimethyl terephthalate. The carboxylic acids may be used either individually or in a mixture. Carboxylic acids used with preference are phthalic acid, terephthalic acid, glutaric acid, adipic acid, sebacic acid and/or succinic acid, particularly preferably adipic acid, phthalic acid and/or succinic acid.

Hydroxycarboxylic acids that may also be used as reaction participants in the preparation of a polyester polyol having terminal hydroxyl groups are for example hydroxycaproic acid, hydroxybutyric acid, hydroxydecanoic acid, hydroxystearic acid, ricinoleic acid and the like. Suitable lactones include caprolactone, butyrolactone and homologs.

Also especially useful for preparation of the polyester polyols are bio-based starting materials and/or derivatives thereof, for example castor oil, polyhydroxy fatty acids, ricinoleic acid, hydroxyl-modified oils, grapeseed oil, black cumin oil, pumpkin seed oil, borage seed oil, soybean oil, wheat germ oil, rapeseed oil, sunflower seed oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, fatty acids, hydroxyl-modified and epoxidized fatty acids and fatty acid esters, for example based on myristoleic acid, palmitoleic acid, oleic acid, vaccenic acid, petroselic acid, gadoleic acid, erucic acid, nervonic acid, linoleic acid, alpha- and gamma-linolenic acid, stearidonic acid, arachidonic acid, timnodonic acid, clupanodonic acid and cervonic acid. Esters of ricinoleic acid with polyfunctional alcohols, for example glycerol, are especially preferred. Preference is also given to the use of mixtures of such bio-based acids with other carboxylic acids, for example phthalic acids.

Cyclic carboxylic anhydrides used may, for example, be phthalic anhydride, maleic anhydride, succinic anhydride, tetrahydrophthalic anhydride, hexahydrophthalic anhydride, glutaric anhydride and/or dodecenylsuccinic anhydride; preference is given to phthalic anhydride, maleic anhydride, succinic anhydride and mixtures of the cyclic carboxylic anhydrides mentioned.

Acyclic esters may, for example, be dimethyl terephthalate, diethyl succinate, ethyl methyl malonate, diethyl malonate and/or diethyl adipate, preference being given to dimethyl terephthalate.

Cyclic esters may, for example, be lactides, or aliphatic or aromatic lactones. The aliphatic or aromatic lactones may, for example, be 4-membered lactones such as β-propiolactone, β-butyrolactone, β-isovalerolactone, β-caprolactone, β-isocaprolactone and β-methyl-τ3-valerolactone, or 5-membered lactones, such as γ-butyrolactone, γ-valerolactone, 5-methylfuran-2(3H)-one, 5-methylidenedihydrofuran-2(3H)-one, 5-hydroxyfuran-2(5H)-one, 2-benzofuran-1(3H)-one and 6-methyl-2-benzofuran-1(3H)-one, or 6-membered lactones, such as δ-valerolactone, 1,4-dioxan-2-one, dihydrocoumarin, 1H-isochromen-1-one, 8H-pyrano[3,4-b]pyridin-8-one, 1,4-dihydro-3H-isochromen-3-one, 7,8-dihydro-5H-pyrano[4,3-b]pyridin-5-one, 4-methyl-3,4-dihydro-1H-pyrano[3,4-b]pyridin-1-one, 6-hydroxy-3,4-dihydro-1H-isochromen-1-one, 7-hydroxy-3,4-dihydro-2H-chromen-2-one, 3-ethyl-1H-isochromen-1-one, 3-(hydroxy methyl)-1H-isochromen-1-one, 9-hydroxy-1H,3H-benzo[de]isochromen-1-one, 6,7-dimethoxy-1,4-dihydro-3H-isochromen-3-one and 3-phenyl-3,4-dihydro-1H-isochromen-1-one, or 7-membered lactones, such as ε-caprolactone, 1,5-dioxepan-2-one, 5-methyloxepan-2-one, oxepane-2,7-dione, thiepan-2-one, 5-chlorooxepan-2-one, (4S)-4-(propan-2-yl)oxepan-2-one, 7-butyloxepan-2-one, 5-(4-aminobutyl)oxepan-2-one, 5-phenyloxepan-2-one, 7-hexyloxepan-2-one, (5S,7S)-5-methyl-7-(propan-2-yl)oxepan-2-one and 4-methyl-7-(propan-2-yl)oxepan-2-one, or lactones with higher numbers of ring members, such as (7E)-oxacycloheptadec-7-en-2-one. Particular preference is given to ε-caprolactone and dihydrocoumarin. Lactides used may, for example, be glycolide (1,4-dioxane-2,5-dione), L-lactide (L-3,6-dimethyl-1,4-dioxane-2,5-dione), D-lactide, DL-lactide, mesolactide and 3-methyl-1,4-dioxane-2,5-dione, 3-hexyl-6-methyl-1,4-dioxane-2,5-diones, 3,6-di(but-3-en-1-yl)-1,4-dioxane-2,5-dione (in each case inclusive of optically active forms). Particular preference is given to using L-lactide.

As well as the diol obtained from the first process stage, it is additionally possible alcohols, for example ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, pentane-1,5-diol, hexane-1,6-diol and isomers, neopentyl glycol or neopentyl glycol hydroxypivalate, polyols such as trimethylolpropane, glycerol, erythritol, pentaerythritol, trimethylolbenzene or trishydroxyethyl isocyanurate. Preference is given to using ethylene glycol, diethylene glycol, butane-1,4-diol, pentane-1,5-diol, hexane-1,6-diol, glycerol and trimethylolpropane, or mixtures of at least two of the alcohols mentioned.

Polyether ester polyols are those compounds containing ether groups, ester groups and OH groups. Organic dicarboxylic acids having up to 12 carbon atoms are suitable for preparing the polyether ester polyols, preferably aliphatic dicarboxylic acids having 4 to 6 carbon atoms or aromatic dicarboxylic acids used individually or in a mixture. Examples include suberic acid, azelaic acid, decanedicarboxylic acid, maleic acid, malonic acid, phthalic acid, pimelic acid and sebacic acid and in particular glutaric acid, fumaric acid, succinic acid, adipic acid, phthalic acid, terephthalic acid and isoterephthalic acid. As well as organic dicarboxylic acids, it is also possible to use cyclic carboxylic anhydrides, acyclic esters and/or cyclic esters. The use of proportions of the aforementioned bio-based starting materials, especially of fatty acids or fatty acid derivatives (oleic acid, soybean oil, etc.) is likewise possible.

A further component used for preparation of the polyetherester polyols is polyether polyols that are obtained by alkoxylating the diol obtained from the first process step and any further H-functional starter substances, such as polyhydric alcohols. As well as the diol obtained from the first process stage, it is additionally possible to use polyhydric alcohols, for example ethane-1,2-diol, propane-1,3-diol, propane-1,2-diol, butane-1,4-diol, pentene-1,5-diol, pentane-1,5-diol, neopentyl glycol, hexane-1,6-diol, heptane-1,7-diol, octane-1,8-diol, decane-1,10-diol, 2-methylpropane-1,3-diol, 2,2-dimethylpropane-1,3-diol, 3-methylpentane-1,5-diol, 2-butyl-2-ethylpropane-1,3-diol, 2-butene-1,4-diol and 2-butyne-1,4-diol, diethylene glycol, triethylene glycol, tetraethylene glycol, dibutylene glycol, tributylene glycol, tetrabutylene glycol, dihexylene glycol, trihexylene glycol, tetrahexylene glycol, diethylene glycol, 1,1,1-trimethylolpropane, triethanolamine, glycerol, sorbitol and pentaerythritol, and polyethylene oxide polyols started from triols or tetraols.

Polyetherester polyols may also be prepared by the alkoxylation, especially by ethoxylation and/or propoxylation, of reaction products that are obtained by the reaction of organic dicarboxylic acids, cyclic carboxylic anhydrides, acyclic esters and/or cyclic esters, and also components with Zerewitinoff-active hydrogens, especially diols and polyols.

Processes for preparing the polyol have been described, for example, by Ionescu in “Chemistry and Technology of Polyols for Polyurethanes”, Rapra Technology Limited, Shawbury 2005, p. 55 ff. (ch. 4: Oligo-Polyols for Elastic Polyurethanes), p. 263 ff. (ch. 8: Polyester Polyols for Elastic Polyurethanes) and especially on p. 321 ff. (ch. 13: Polyether Polyols for Rigid Polyurethane Foams) and p. 419 ff. (ch. 16: Polyester Polyols for Rigid Polyurethane Foams). It is also possible to obtain polyester polyols and polyether polyols by glycolysis of suitable polymer recyclates. Suitable polyethercarbonate polyols and the preparation thereof are described, for example, in EP 2 910 585 A1, [0024]-[0041].

Examples of polycarbonate polyols and the preparation thereof can be found, inter alia, in EP 1 359 177 A1. The preparation of suitable polyetherester polyols has been described, inter alia, in WO 2010/043624 A and in EP 1 923 417 A.

In a first embodiment, the invention relates to a process for preparing diol, characterized in that the process comprises the steps of
(1-i) adding alkylene oxide and carbon dioxide onto an H-functional starter substances in the presence of a catalyst to obtain polyethercarbonate polyol and cyclic carbonate,
(1-ii) separating the cyclic carbonate from the resulting reaction mixture from step (1-i),
(1-iii) hydrolyzing the cyclic carbonate separated from step (1-ii) to carbon dioxide and diol,
(1-iv) optionally purifying the diol from step (1-iii) by distillation,
wherein
(η) a Lewis or Brønsted acid, excluding carboxylic acids having a pKa of >3.0, and optionally water are added to the cyclic carbonate from step (1-ii) and/or the diol, and the reaction mixture obtained is optionally neutralized.

In a second embodiment, the invention relates to a process according to the first embodiment, characterized in that, in step (1-i), the addition is effected in the presence of a double metal cyanide catalyst or a metal complex catalyst based on the metals zinc and/or cobalt.

In a third embodiment, the invention relates to a process according to the first or second embodiment, characterized in that, in step (1-i),
(α) a reactor is charged with a portion of H-functional starter substance and/or a suspension medium having no H-functional groups, optionally together with catalyst,
(β) a DMC catalyst is activated if appropriate by adding a portion (based on the total amount of alkylene oxide used in the activation and copolymerization) of alkylene oxide to the mixture from step (α), where this addition of a portion of alkylene oxide can optionally be effected in the presence of CO₂, and in which case the temperature spike (“hotspot”) that occurs owing to the exothermic chemical reaction that follows and/or a pressure drop in the reactor is then awaited in each case, and where step (β) for activation may also be effected repeatedly,
(γ) an H-functional starter substance, alkylene oxide and optionally a suspension medium having no H-functional groups and/or carbon dioxide are metered into the reactor during the reaction,
(δ) the reaction mixture removed continuously in step (γ) is optionally transferred into a postreactor in which, by way of a postreaction, the content of free alkylene oxide in the reaction mixture is reduced.

In a fourth embodiment, the invention relates to a process according to any of embodiments 1 to 3, characterized in that, in step (1-ii), the cyclic carbonate is separated off by thermal methods.

In a fifth embodiment, the invention relates to a process according to any of embodiments 1 to 4, characterized in that the Lewis or Brønsted acid in step (η) is selected from at least one compound from the group consisting of HCl, HBr, HI, H₂SO₄, H₂SO₃, H₃PO₄, H₃PO₃, HNO₃, carboxylic acids having a pKa ≤3.0, compounds having sulfonic acid groups and metal salts.

In a sixth embodiment, the invention relates to a process according to any of embodiments 1 to 5, characterized in that, in step (1-ii), the cyclic carbonate is selected from at least one compound from the group consisting of cyclic propylene carbonate and cyclic ethylene carbonate.

In a seventh embodiment, the invention relates to a process according to any of embodiments 1 to 6, characterized in that the cyclic carbonate obtained in step (1-ii) is purified by distillation prior to step (1-iii).

In an eighth embodiment, the invention relates to a process according to any of embodiments 1 to 7, characterized in that, in step (1-iii), the hydrolysis catalyst used is at least one compound selected from the group consisting of alkali metal hydroxides, alkaline earth metal hydroxides and hydrolases.

In a ninth embodiment, the invention relates to a process according to the eighth embodiment, characterized in that, in step (1-iii), the hydrolysis catalyst used is at least one compound selected from the group consisting of the alkali metal hydroxides.

In a tenth embodiment, the invention relates to a process according to any of embodiments 1 to 9, characterized in that 0.05% to 1% by weight, based on the cyclic carbonate used in step (1-iii), of a hydrolysis catalyst is used.

In an eleventh embodiment, the invention relates to a process according to any of embodiments 1 to 10, characterized in that step (1-iii) is performed at a temperature of at least 40° C.

In a twelfth embodiment, the invention relates to a process according to any of embodiments 1 to 11, characterized in that, in step (1-iii), the molar ratio of cyclic carbonate to water is 1:1 to 1:10.

In a thirteenth embodiment, the invention relates to a process according to any of embodiments 1 to 12, characterized in that a step (1-iv) is performed.

In a fourteenth embodiment, the invention relates to a process for preparing polyol, characterized in that
(2-i) polyol is obtained by
   a) adding alkylene oxide and optionally carbon dioxide, cyclic carboxylic anhydride and/or cyclic esters onto a diol obtained by a process according to any of embodiments 1 to 13 and optionally further H-functional starter substance
      or
   b) reacting carboxylic acid, cyclic carboxylic anhydride, acyclic ester and/or cyclic ester with a diol obtained by a process according to any of embodiments 1 to 13 and optionally further alcohols.

In a fifteenth embodiment, the invention relates to a process according to the fourteenth embodiment, characterized in that the polyol in step (2-i) is selected from at least one compound from the group consisting of polyether polyol, polyester polyol, polyetherester polyol and polyethercarbonate polyol.

In a sixteenth embodiment, the invention relates to a process according to the thirteenth embodiment, characterized in that step (1-iv) is performed at a pressure of 150 mbar or lower.

In a seventeenth embodiment, the invention relates to a process according to any of embodiments 1 to 13, characterized in that, in step (1-ii), a thin-film evaporator or a falling-film evaporator is used in combination with a stripping column.

### EXAMPLES

The OH number (hydroxyl number) was determined in accordance with DIN 53240-2 (November 2007).

Viscosity was determined on an Anton Paar Physica MCR 501 rheometer. A cone-plate configuration having a separation of 1 mm was selected (DCP25 measurement system). The polyethercarbonate polyol (0.1 g) was applied to the rheometer plate and subjected to a shear of 0.01 to 1000 l/s at 25° C. and the viscosity was measured every 10 s for 10 min. The FIGURE reported is the viscosity averaged over all measurement points.

The proportion of CO₂ incorporated in the resulting polyethercarbonate polyol and the ratio of propylene carbonate to polyethercarbonate polyol were determined by means of ¹H NMR (Bruker DPX 400, 400 MHz; zg30 pulse program, relaxation delay d1: 10 s, 64 scans). Each sample was dissolved in deuterated chloroform. The relevant resonances in the ¹H NMR (based on TMS=0 ppm) are as follows:

cyclic carbonate (formed as a by-product) resonance at 4.5 ppm, carbonate resulting from carbon dioxide incorporated in the polyethercarbonate polyol (resonances at 5.1 to 4.8 ppm), unreacted propylene oxide (PO) having a resonance at 2.4 ppm, polyether polyol (i.e. without incorporated carbon dioxide) having resonances at 1.2 to 1.0 ppm.

The mole fraction of the carbonate incorporated in the polymer in the reaction mixture is calculated as per formula (VIII) as follows, the following abbreviations being used:
A(4.5)=area of the resonance at 4.5 ppm for cyclic carbonate (corresponds to one hydrogen atom)
A(5.1−4.8)=area of the resonance at 5.1−4.8 ppm for polyethercarbonate polyol and one hydrogen atom for cyclic carbonate
A(2.4)=area of the resonance at 2.4 ppm for free, unreacted PO
A(1.2−1.0)=area of the resonance at 1.2−1.0 ppm for polyether polyol

Taking account of the relative intensities, the values for the polymer-bound carbonate (“linear carbonate” LC) in the reaction mixture were converted to mol % by the following formula (VIII):

$$LC = \frac{A ( 5.1 - 4.8 ) - A ( 4.5 )}{A ( 5.1 - 4.8 ) + A ( 2.4 ) + 0.33 * A ( 1.2 - 1.0 )} * 100 \qquad ( VIII )$$

The proportion by weight (in % by weight) of polymer-bound carbonate (LC′) in the reaction mixture was calculated by formula (IX)

$${LC}^{′} = \frac{[ A ( 5.1 - 4.8 ) - A ( 4.5 ) ] * 102}{N} * 100 \% \qquad ( IX )$$

where the value of D (“denominator” D) is calculated by formula (X):

D[A(5.1−4.8)−A(4.5)]*102+A(4.5)*102+A(2.4)*58+0.33*A(1.2−1.0)*58 (X)

The factor of 102 results from the sum of the molar masses of CO₂ (molar mass 44 g/mol) and of propylene oxide (molar mass 58 g/mol); the factor of 58 results from the molar mass of propylene oxide.

The proportion by weight (in % by weight) of cyclic carbonate (CC′) in the reaction mixture was calculated by formula (XI)

$${CC}^{′} = \frac{A ( 4.5 ) * 102}{N} * 100 \% \qquad ( XI )$$

where the value of D is calculated by formula (X).

In order to calculate the composition based on the polymer component (consisting of polyether which has been formed from propylene oxide during the activation steps which optionally take place under CO₂-free conditions, and polyethercarbonate polyol formed from starter, propylene oxide and carbon dioxide during the activation steps which take place in the presence of CO₂ and during the copolymerization) from the values for the composition of the reaction mixture, the non-polymeric constituents of the reaction mixture (i.e. cyclic propylene carbonate and any unconverted propylene oxide present) were mathematically eliminated. The proportion by weight of the repeat carbonate units in the polyethercarbonate polyol was converted to a proportion by weight of carbon dioxide using the factor A=44/(44+58). The FIGURE for the CO₂ content in the polyethercarbonate polyol (see PECP-1) is normalized to the polyethercarbonate polyol molecule formed in the copolymerization and the activation steps.

The amount of cyclic propylene carbonate formed is determined via the mass balance of the total amount of cyclic propylene carbonate present in the reaction mixture. The total amount of cyclic propylene carbonate is found from the quantitative separation of cyclic propylene carbonate from the reaction mixture by means of two-stage thermal workup (falling-film evaporator and nitrogen stripping column).

The proportions of spiro-orthocarbonate and DMD were determined using gas chromatography coupled to mass spectrometry (Agilent 6890/5973), wherein the analyte was used in non-derivatized form. Detection was effected by means of a flame ionization detector. Quantification was effected by area integration.

Starting Materials:
PECP-1: polyethercarbonate polyol having a functionality of 2.8, an OH number of 56 mg KOH/g and a CO₂ content of 20% by weight
Glycerol: from August Hedinger GmbH & Co. KG
Propylene glycol: from August Hedinger GmbH & Co. KG
Adipic acid: from BASF SE
Antioxidant: Irganox® 1076, (from BASF SE)
p-TS: p-toluenesulfonic acid monohydrate (from Sigma-Aldrich)
Amberlyst 15: acidic ion exchanger containing sulfonic acid groups (from Sigma-Aldrich)
Amberlyst 36: acidic ion exchanger containing sulfonic acid groups (from Sigma-Aldrich)
Conc. HCl: 6M hydrochloric acid (from Sigma-Aldrich)
NaHCO₃: sodium hydrogencarbonate (from Sigma-Aldrich)
MgSO₄: magnesium sulfate (from Sigma-Aldrich)
SnCl₂: tin(II) chloride (from Sigma-Aldrich)

The DMC catalyst used in all examples was DMC catalyst prepared according to example 6 in WO 01/80994 A1.

### Example 1a: Preparation and Separation of Cyclic Propylene Carbonate (cPC) from the Addition of Propylene Oxide and Carbon Dioxide onto H-Functional Starter Substance

Step (1-i):
(α) A continuously operated 60 L pressure reactor with gas metering device and product discharge tube was initially charged with 32.9 L of PECP-1 containing 200 ppm of DMC catalyst.
(γ) At a temperature of 108° C. and a total pressure of 65 bar (absolute), the following components were metered in at the metering rates specified while stirring (11 Hz):
   propylene oxide at 7.0 kg/h
   carbon dioxide at up to 2.3 kg/h, such that the pressure of 65 bar is kept constant,
   mixture of glycerol/propylene glycol (85% by weight/15% by weight) containing 0.69% by weight (based on the mixture of glycerol and propylene glycol) of DMC catalyst (unactivated) and 146 ppm (based on the mixture of glycerol, propylene glycol and DMC catalyst) of H₃PO₄ (used in the form of an 85% aqueous solution) at 0.27 kg/h.
   The reaction mixture was withdrawn continuously from the pressure reactor via the product discharge tube, such that the reaction volume (32.9 L) was kept constant, with the average dwell time of the reaction mixture in the reactor of 200 min.
(δ) To complete the reaction, the reaction mixture withdrawn was transferred into a postreactor (tubular reactor having a reaction volume of 2.0 L) which had been heated to 120° C. The average dwell time of the reaction mixture in the postreactor was 12 min.
   The product was then decompressed to atmospheric pressure and then 500 ppm of antioxidant (Irganox® 1076) was added.
   Thereafter, to ascertain the selectivity (cyclic/linear carbonate ratio) of the reaction mixture downstream of the postreactor, a sample was taken and the content of cyclic and linear carbonate was determined by means of ¹H NMR analysis.
   Subsequently, the product was brought to a temperature of 120° C. by means of a heat exchanger and immediately thereafter transferred to a 332 L tank and kept at the temperature of 120° C. for a dwell time of 4 hours.
   On conclusion of the dwell time, the product was admixed with 40 ppm of phosphoric acid (component K).

Step (1-ii):

Finally, the reaction mixture obtained from step (1-i), for removal of the cyclic propylene carbonate, was subjected to a two-stage thermal workup, namely in a first stage by means of a falling-film evaporator, followed, in a second stage, by a stripping column operated in a nitrogen countercurrent.

The falling-film evaporator was operated here at a temperature of 160° C. and a pressure of 10 mbar (absolute). The falling-film evaporator used consisted of glass with an exchange area of 0.5 m². The apparatus had an externally heated tube with a diameter of 115 mm and a length of about 1500 mm.

The nitrogen stripping column was operated at a temperature of 160° C., a pressure of 80 mbar (absolute) and a nitrogen flow rate of 0.6 kg N₂/kg product. The stripping column used was a DN80 glass column filled to a height of 8 m with random packings (Raschig #0.3 Super-Rings).

The resultant crude cPC product contains 2000 ppm of spiro-orthocarbonate and 10 900 ppm of dimethyldioxane (DMD).

### Example 1b: Preparation and Separation of cPC from the Addition of Propylene Oxide and Carbon Dioxide onto H-Functional Starter Substance with Subsequent Purification of the cPC

The same process parameters as in example 1 are employed. However, the resultant crude cPC product is subsequently purified by distillatively removing 5% by weight of the starting material at 10 mbar and a bottom temperature of 115° C.

The crude cPC product contains 1446 ppm of spiro-orthocarbonate and <50 ppm of dimethyldioxane.

### Example 2: Removal of Spiro-Orthocarbonate by Means of Adipic Acid

(η) A 1 L 4-neck glass flask, equipped with mechanical stirrer system, thermometer and jacketed coil reflux condenser, is initially charged with 250 g of the crude cPC product from example 1a and 0.6 g of adipic acid, and the mixture is stirred at 100° C. for 1 hour. The composition was determined by neutralizing with NaHCO₃ and drying over MgSO₄. The reaction mixture was analyzed by GC-MS.

### Example 3: Removal of Spiro-Orthocarbonate by Means of Ion Exchanger

(η) A 1 L 4-neck glass flask, equipped with mechanical stirrer system, thermometer and jacketed coil reflux condenser, was initially charged with 250 g of the crude cPC product from example 1a and 5 g of Amberlyst 15, and the mixture is stirred at 50° C. for 1 hour. The reaction mixture is filtered and analyzed by means of GC-MS.

### Example 4: Removal of Spiro-Orthocarbonate by Means of Ion Exchanger

(η) A 1 L 4-neck glass flask, equipped with mechanical stirrer system, thermometer and jacketed coil reflux condenser, is initially charged with 250 g of the crude cPC product from example 1a and 5 g of Amberlyst 36, and the mixture is stirred at 50° C. for 1 hour. The reaction mixture is filtered and analyzed by means of GC-MS.

### Example 5: Removal of Spiro-Orthocarbonate by Means of HCl

(η) A 1 L 4-neck glass flask, equipped with mechanical stirrer system, thermometer and jacketed coil reflux condenser, is initially charged with 250 g of the crude cPC product from example 1a and 7.6 ml of conc. HCl, and the mixture is stirred at 25° C. for 1 hour. The composition was determined by neutralizing with NaHCO₃ and drying over MgSO₄. The reaction mixture was analyzed by GC-MS.

### Example 6: Removal of Spiro-Orthocarbonate by Means of SnC12

(η) A 1 L 4-neck glass flask, equipped with mechanical stirrer system, thermometer and jacketed coil reflux condenser, is initially charged with 250 g of the crude cPC product from example 1a and 1.7 g of SnC12, and the mixture is stirred at 50° C. for 1 hour. The composition was determined by neutralizing with NaHCO₃ and drying over MgSO₄. The reaction mixture was analyzed by GC-MS.

### Example 7: Removal of Spiro-Orthocarbonate by Means of p-TS

(η) A 1 L 4-neck glass flask, equipped with mechanical stirrer system, thermometer and jacketed coil reflux condenser, is initially charged with 250 g of the product from example 1b together with 1.44 g of p-TS, and the mixture is stirred at 25° C. for 1 hour. The reaction mixture was analyzed by GC-MS.

### Example 8: Removal of Spiro-Orthocarbonate by Means of p-TS

(η) A 1 L 4-neck glass flask, equipped with mechanical stirrer system, thermometer and jacketed coil reflux condenser, is initially charged with 600 g of the product from example 1b together with 6 g of p-TS, and the mixture is stirred at 100° C. for 1 hour. The reaction mixture was analyzed by GC-MS.

### Example 9: Removal of Spiro-Orthocarbonate by Means of p-TS

(η) A 1 L 4-neck glass flask, equipped with mechanical stirrer system, thermometer and jacketed coil reflux condenser, is initially charged with 174 g of the product from example 1b together with 2 g of p-TS and 26 g of demineralized water, and the mixture is stirred at 100° C. for 1 hour. The reaction mixture was analyzed by GC-MS.

TABLE 1
Analyses of the acid-treated crude cPC products

| | | Example: | | | | | | | |
|---|---|---|---|---|---|---|---|---|---|
| | | 2 | 3 | 4 | 5 | 6 | 7 | 8 | 9 |
| Crude cPC product | g | 250 | 250 | 250 | 250 | 250 | 250 | | |
| from example 1a | | | | | | | | | |
| Crude cPC product | g | | | | | | | 600 | 174 |
| from example 1b | | | | | | | | | |
| Adipic acid | g | 0.6 | | | | | | | |
| Amberlyst 15 | g | | 5 | | | | | | |
| Amberlyst 36 | g | | | 5 | | | | | |
| HCl | ml | | | | 7.6 | | | | |
| SnCl₂ | g | | | | | 1.7 | | | |
| p-TS | g | | | | | | 1.44 | 6 | 2 |
| Water | g | | | | | | | | 26 |
| Reaction temperature | ° C. | 100 | 50 | 50 | 25 | 50 | 25 | 100 | 100 |
| Reaction time | h | 1 | 1 | 1 | 1 | 1 | 1 | 1 | 1 |
| Impurities before reaction: | | | | | | | | | |
| Spiro-orthocarbonate | ppm | 2000 | 2000 | 2000 | 2000 | 2000 | 2000 | 1446 | 1446 |
| DMD | ppm | 10900 | 10900 | 10900 | 10900 | 10900 | 10900 | <50 | <50 |
| Impurities after reaction: | | | | | | | | | |
| Spiro-orthocarbonate | ppm | 2000 | <50 | <50 | <50 | <50 | <50 | <50 | <50 |
| DMD | ppm | 7590 | 9427 | 10520 | 10570 | 10330 | 10700 | 5884 | <50 |

Table 1 shows that the proportion of spiro-orthocarbonate in examples 3 to 9 is reduced to less than 50 ppm in the presence of acid catalysts.

Example 9 shows that, in a preferred embodiment of the process of the invention, the formation of DMD is simultaneously suppressed.

### Example 10: Hydrolytic Cleavage of the Cyclic Carbonate

cPC that has been obtained by the aforementioned process of the invention and has been freed of spiro-orthocarbonate was subjected to alkaline hydrolysis as described below:

Step (1-iii):

A 1 L 4-neck glass flask, equipped with mechanical stirrer system, thermometer and jacketed coil reflux condenser with downstream gasmeter, was initially charged with 522 g (5.1 mol) of the cPC freed of spiro-orthocarbonate from example 8, table 1, and 2.25 g of KOH.

At a pressure of 11 mbar and a temperature of 105° C., DMD present was distilled off. Subsequently, 180 g of demineralized water was added and the solution was heated to reflux while stirring, with elimination of CO₂. The reaction was continued under the aforementioned reaction conditions until the evolution of CO₂ had ceased completely.

The reaction mixture was fractionally distilled, using a column having random packing (about 50 cm), a distillation system and a vacuum membrane pump for the removal of water.

On completion of the removal of water, without the column having random packing, 1,2-propylene glycol was distilled off using a distillation system, with a distillation bottom temperature of 90° C. and a top temperature of 85° C. at a vacuum of 10 mbar.

199 g (2.6 mol) of 1,2-propylene glycol was distilled off in the main fraction, and 39 g of residue was left in the flask.

Analysis of the 1,2-Propylene Glycol Obtained:

Hydroxyl number: 1446 mg KOH/g (theor.; 1474 mg KOH/g) DMD content: <50 ppm (GC-MS)

Spiro-orthocarbonate: <50 ppm (GC-MS)

### Example 11: Hydrolysis of cPC without Removal of Spiro-Orthocarbonate

The breakdown of the spiro-orthocarbonate may also follow the hydrolysis of the cPC, i.e. proceed from distilled 1,2-propylene glycol:

Step (1-iii):

A 10 L 4-neck glass flask equipped with a mechanical stirrer, thermometer and jacketed coil reflux condenser with downstream gasmeter was initially charged with 6630 g (65 mol) of the crude cPC product from example 1b and a solution of 2340 g (130 mol) of demineralized water and 16.6 g of KOH. The solution was heated to reflux while stirring, with elimination of CO₂. Once the evolution of CO₂ had distinctly slowed (after the formation of 1030 L of CO₂), 1530 g (15 mol) of the crude cPC product from example 1b was metered in and the reaction was continued under the aforementioned reaction conditions until the evolution of CO₂ had ceased completely.

Step (1-iv):

The reaction mixture that was obtained from step (1-iii) and comprised 1,2-propylene glycol was fractionally distilled, with removal of water using a column having random packing (about 50 cm), a distillation apparatus and a vacuum membrane pump.

On completion of removal of the water, without the randomly packed column, 1,2-propylene glycol was distilled off using a distillation apparatus, with the temperature of the distillation bottom at 90° C. and at a top temperature at 85° C. at a vacuum of 10 mbar.

5205 g (68.5 mol) of 1,2-propylene glycol was distilled off in the main fraction, and 875 g of residue was left in the flask. The latter contains the KOH used and can be used for further hydrolysis reactions.

Analysis of the 1,2-Propylene Glycol Obtained:

Purity: 97.6% (GC-MS)

DMD content: <50 ppm

Spiro-orthocarbonate: 550 ppm

### Example 12: Removal of Spiro-Orthocarbonate after Step (1-iv)

(η) A 1 L 4-neck glass flask, equipped with mechanical stirrer system, thermometer and jacketed coil reflux condenser, is initially charged with 174 g of the 1,2-propylene glycol obtained from example 11 together with 1 g of p-TS and 5 g of demineralized water, and the mixture was stirred at 100° C. for 1 hour. The reaction mixture was analyzed by GC-MS.

TABLE 2
Analyses of the acid-treated crude cPC product after step (1-iv)

| Example: | | 12 |
|---|---|---|
| 1,2-Propylene glycol | g | 174 |
| (main fraction after the | | |
| aforementioned CPC hydrolysis) | | |
| Water | g | 5 |
| p-TS | g | 1 |
| Reaction temperature | ° C. | 100 |
| Reaction time | h | 1 |

Impurities before reaction:

| | | |
|---|---|---|
| Spiro-orthocarbonate | ppm | 550 |
| DMD | ppm | <50 |

Impurities after reaction:

| | | |
|---|---|---|
| Spiro-orthocarbonate | ppm | <50 |
| DMD | ppm | <50 |

1. A process for preparing diol, the process comprises the steps of:
(1-i) adding alkylene oxide and carbon dioxide onto an H-functional starter substance in the presence of a catalyst to obtain polyethercarbonate polyol and cyclic carbonate,
(1-ii) separating the cyclic carbonate from the resulting reaction mixture from step (1-i), and
(1-iii) hydrolyzing the cyclic carbonate separated from step (1-ii) to carbon dioxide and diol,
wherein
(η) a Lewis or Brønsted acid, excluding carboxylic acids having a pKa of >3.0, is added to the cyclic carbonate from step (1-ii) and/or the diol.

2. The process as claimed in claim 1, wherein in step (1-i), the addition is effected in the presence of a double metal cyanide catalyst or a metal complex catalyst based on the metals zinc and/or cobalt.

3. The process as claimed in claim 1, wherein in step (1-i),
(α) a reactor is charged with a portion of H-functional starter substance and/or a suspension medium having no H-functional groups,
(γ) an H-functional starter substance, alkylene oxide are metered into the reactor during the reaction.

4. The process as claimed in claim 1, wherein in step (1-ii), the cyclic carbonate is separated off by thermal methods.

5. The process as claimed in claim 1, wherein the Lewis or Brønsted acid in step (η) is selected from at least one compound from the group consisting of HCl, HBr, HI, H₂SO₄, H₂SO₃, H₃PO₄, H₃PO₃, HNO₃, carboxylic acids having a pKa≤3.0, compounds having sulfonic acid groups and metal salts.

6. The process as claimed in claim 1, wherein in step (1-ii), the cyclic carbonate is selected from at least one compound from the group consisting of cyclic propylene carbonate and cyclic ethylene carbonate.

7. The process as claimed in claim 1, wherein the cyclic carbonate obtained in step (1-ii) is purified by distillation prior to step (1-iii).

8. The process as claimed in claim 1, wherein in step (1-iii), the hydrolysis catalyst used is at least one compound selected from the group consisting of alkali metal hydroxides, alkaline earth metal hydroxides and hydrolases.

9. The process as claimed in claim 8, wherein in step (1-iii), the hydrolysis catalyst used is at least one compound selected from the group consisting of the alkali metal hydroxides.

10. The process as claimed in claim 1, wherein 0.05% to 1% by weight, based on the cyclic carbonate used in step (1-iii), of a hydrolysis catalyst is used.

11. The process as claimed in claim 1, wherein step (1-iii) is performed at a temperature of at least 40° C.

12. The process as claimed in claim 1, wherein in step (1-iii), the molar ratio of cyclic carbonate to water is 1:1 to 1:10.

13. The process as claimed in claim 1, wherein the process further comprises:
(1-iv) purifying the diol from step (1-iii) by distillation.

14. A process for preparing polyol, wherein
(2-i) polyol is obtained by
a) adding alkylene oxide onto a diol obtained by a process as claimed in claim 1 and
or
b) reacting carboxylic acid, cyclic carboxylic anhydride, acyclic ester and/or cyclic ester with a diol obtained by a process as claimed in claim 1.

15. The process as claimed in claim 14, wherein the polyol in step (2-i) is selected from at least one compound from the group consisting of polyether polyol, polyester polyol, polyetherester polyol and polyethercarbonate polyol.

16. The process as claimed in claim 1, wherein step (ii) further comprises adding water to the cyclic carbonate.

17. The process as claimed in claim 1, wherein in step (ii), the reaction mixture obtained is neutralized.

18. The process as claimed in claim 3,
wherein in step (α) the reactor is charged with the portion of H-functional starter substance and/or the suspension medium having no H-functional groups together with catalyst,
wherein in step (γ) a suspension medium having no H-functional groups and/or carbon dioxide are metered into the reactor during the reaction,
and
wherein in step (1-i),
(β) a DMC catalyst is activated by adding a portion (based on the total amount of alkylene oxide used in the activation and copolymerization) of alkylene oxide to the mixture from step (α), where this addition of a portion of alkylene oxide is effected in the presence of CO₂, and in which case the temperature spike that occurs owing to an exothermic chemical reaction that follows and/or a pressure drop in the reactor is then awaited in each case, and where step (β) for activation is effected repeatedly, and
(δ) the reaction mixture removed continuously in step (γ) is transferred into a postreactor in which, by way of a postreaction, the content of free alkylene oxide in the reaction mixture is reduced.

19. The process as claimed in claim 14, wherein step (a) further comprises adding carbon dioxide, cyclic carboxylic anhydride and/or cyclic esters onto the diol.

20. The process as claimed in claim 14, wherein in step (a) the alkylene oxide is added onto the diol and further H-functional starter substance.
